(19) United States
(12) Patent Application Publication
(10) Pub. No.: US 2024/0346671 A1
(43) Pub. Date: Oct. 17, 2024

(54) DEFORMABLE IMAGE REGISTRATION

(71) Applicant: Elekta Limited, Crawley (GB)

(72) Inventors: Wenzhe YIN, Crawley (GB); Jan-Jakob SONKE, Crawley (GB); Efstratios GAVVES, Crawley (GB)

(21) Appl. No.: 18/634,690

(22) Filed: Apr. 12, 2024

(30) Foreign Application Priority Data
Apr. 13, 2023 (GB) 2305467.9

(51) Int. Cl. G06T 7/37; A61N 5/10; G06T 3/18; G06T 3/4046; G16H 20/40

(52) U.S. Cl. CPC G06T 7/37; A61N 5/1039; G06T 3/18; G06T 3/4046; G16H 20/40; G06T 2207/20016; G06T 2207/20021; G06T 2207/20081; G06T 2207/20084; G06T 2207/30004

(57) ABSTRACT

A method for performing deformable image registration on first and second volumetric medical images comprises (i) for each image, extracting features at each of a plurality of different scales, (ii) initiating a mapping between the first and second volumetric medical images at the lowest of the plurality of different scales, and (iii) sequentially, for each scale of the plurality of different scales that is above the lowest scale, predicting a mapping between the first and second volumetric medical images at a given scale, and correcting the predicted mapping between the first and second volumetric medical images at the given scale. The method further comprises (iv) predicting the deformation field between the first and second volumetric medical images at full resolution using the corrected prediction of the mapping at the highest of the plurality of different scales.

### CLAIM FOR PRIORITY

This application claims the benefit of priority of British Application No. 2305467.9, filed Apr. 13, 2023, which is incorporated by reference in its entirety.

### TECHNICAL FIELD

The present disclosure relates to a method for performing deformable image registration on first and second volumetric medical images. The method may be performed by an image registration node. The present disclosure also relates to an image registration node, and to a computer program product configured, when run on a computer, to carry out a method for performing deformable image registration on first and second volumetric medical images.

### BACKGROUND

Deformable image registration is an important task in medical image analysis, with the objective of finding geometric correspondence between two images. One example application of deformable image registration is in the delivery of Radiotherapy, which may be used to treat cancers or other conditions in human or animal tissue. The treatment planning procedure for radiotherapy may include using a three-dimensional image of the patient to identify a target region, for example the tumour, and to identify organs near the tumour, termed Organs at Risk (OARs). A treatment plan aims to ensure delivery of a required dose of radiation to the tumour, while minimising the risk to nearby OARs. A treatment plan for a patient may be generated in an offline manner, using medical images that have been obtained according to one or more imaging techniques. Such techniques may include for example x-ray, computed tomography (CT), magnetic resonance (MR), etc. The radiation treatment plan includes parameters specifying the direction, cross sectional shape, and intensity of each radiation beam to be applied to the patient. The radiation treatment plan may include dose fractioning, in which a sequence of radiation treatments is provided overt a predetermined period of time, with each treatment delivering a specified fraction of the total prescribed dose. Multiple patient images may be available during the course of radiotherapy treatment, and patient anatomy may frequently change (deform) between delivery of individual dose fractions. Deformable image registration is consequently a useful tool both during planning and delivery of radiotherapy treatments.

### SUMMARY

Given an image pair, some methods for deformable image registration iteratively optimize transformation parameters by minimizing a similarity function between a warped moving image and a fixed image. This classical optimization process is time-consuming and undesirable when fast registration is required, such as may be the case in Adaptive Radiotherapy, or other applications. As an alternative to classical methods, deep neural networks have been widely investigated in the context of deformable medical image registration. An advantage of using deep neural networks for this task is the possibility to exploit representation learning, that is learning high-level features of images from data, in order to then determine geometric correspondence between the images. Strong feature representations can be highly beneficial to support accurate deformable image registration. Another advantage of deep neural networks is speed, with the inference time of learning-based models being more compatible with the requirements of many medical applications.

An example of a learning-based methods for deformable image registration is VoxelMorph (Balakrishnan et al., 2019). This process uses a U-Net architecture (Ronneberger et al., 2015) to estimate the displacement vector field (DVF) for a pair of images, and trains the model in an end-to-end fashion. VoxelMorph is an effective and flexible framework, and can be extended to diffeomorphic and probabilistic registration (Dalca et al., 2019). However, the performance of VoxelMorph is degraded when dealing with large deformations between images. This performance degradation is a consequence of the high degrees of freedom in the transformation parameters (Mok and Chung, 2020). TransMorph (Chen et al., 2022) improves VoxelMorph by using Swin Transformer (Liu et al., 2021) blocks to model the spatial correspondences between the moving and fixed images instead of convolutions. However, recent studies (Pegios and Czolbe, 2022) have shown that TransMorph still faces challenges with large displacements between images. Proposals have been made (in Ru″haak et al. (2017) and Heinrich and Hansen (2020)) to address the large displacement problem by considering the sparse keypoints correspondence and discrete search space. However, achieving high performance deformable image registration, particularly for image pairs which large deformations between image, remains an ongoing challenge.

The present disclosure discusses a method, a registration node, and a computer program product which at least partially address one or more of the challenges mentioned above. It is a further aim of the present disclosure to provide a method, a registration node, and a computer program product which cooperate to perform deformable image registration of volumetric medical images that achieves improved performance for large deformation images when compared with existing methods.

According to a first aspect of the present disclosure, there is provided a method for performing deformable image registration on first and second volumetric medical images. The method comprises (i) for each of the first and second volumetric medical images, extracting features from the images at each of a plurality of different scales, and (ii) initiating a mapping between the first and second volumetric medical images at the lowest of the plurality of different scales. The method further composites (iii) sequentially, for each scale of the plurality of different scales that is above the lowest scale, predicting a mapping between the first and second volumetric medical images at a given scale using the deformation field and features extracted from the first and second volumetric medical images at the scale immediately below the given scale, and correcting the predicted mapping between the first and second volumetric medical images at the given scale using features extracted from the first and second volumetric medical images at the given scale. The method further comprises (iv) predicting the deformation field between the first and second volumetric medical images at full resolution using the corrected prediction of the mapping at the highest of the plurality of different scales.

According to another aspect of the present disclosure, there is provided a computer implemented method for adaptation of a reference Radiotherapy (RT) treatment plan, wherein the reference RT treatment plan is associated with a first volumetric medical image of a patient. The method comprises acquiring a second volumetric medial image of a patient, and performing deformable image registration of the first and second volumetric medical images using a method according to any one or more of the aspects of examples presented herein. The method further comprises using the predicted deformation field between the first and second volumetric medical images at full resolution to adapt the reference treatment plan.

According to another aspect of the present disclosure, there is provided a computer program product comprising a computer readable non-transitory medium, the computer readable medium having computer readable code embodied therein, the computer readable code being configured such that, on execution by a suitable computer or processor, the computer or processor is caused to perform a method according to any one of the aspects or examples of the present disclosure.

According to another aspect of the present disclosure, there is provided a registration node for performing deformable image registration on first and second volumetric medical images. The registration node comprises processing circuitry configured to cause the registration node to (i) for each of the first and second volumetric medical images, extract features from the images at each of a plurality of different scales, and (ii) initiate a mapping between the first and second volumetric medical images at the lowest of the plurality of different scales. The processing circuitry is further configured to case the registration node to (iii) sequentially, for each scale of the plurality of different scales that is above the lowest scale, predict a mapping between the first and second volumetric medical images at a given scale using the deformation field and features extracted from the first and second volumetric medical images at the scale immediately below the given scale, and correct the predicted mapping between the first and second volumetric medical images at the given scale using features extracted from the first and second volumetric medical images at the given scale. The processing circuitry is further configured to case the registration node to (iv) predict the deformation field between the first and second volumetric medical images at full resolution using the corrected prediction of the mapping at the highest of the plurality of different scales.

According to another aspect of the present disclosure, there is provided radiotherapy treatment apparatus comprising a registration node according to any one of the aspects or examples of the present disclosure.

According to another aspect of the present disclosure, there is provided planning node for adapting a reference Radiotherapy (RT) treatment plan, wherein the reference RT treatment plan is associated with a first volumetric medical image of a patient. The planning node comprises processing circuitry configured to cause the planning node to acquire a second volumetric medial image of a patient, and to perform deformable image registration of the first and second volumetric medical images using a method according to any one of the aspects or examples of the present disclosure. The processing circuitry is further configured to cause the planning node to use the predicted deformation field between the first and second volumetric medical images at full resolution to adapt the reference treatment plan.

According to another aspect of the present disclosure, there is provided radiotherapy treatment apparatus comprising a planning node according to any one of the aspects or examples of the present disclosure.

Aspects of the present disclosure thus provide a method and registration node that use a multi scale approach to perform deformable image registration, with features being extracted at a plurality of different scales. Aspects of the present disclosure also employ a prediction-correction framework, in which a mapping between the first and second volumetric medical images is progressively predicted and then corrected at increasing scales. As discussed later in the present disclosure, this multi-scale, prediction-correction approach achieves significantly improved image registration, both for large and small deformation.

### BRIEF DESCRIPTION OF THE DRAWINGS

For a better understanding of the present disclosure, and to show more clearly how it may be carried into effect, reference will now be made, by way of example, to the following drawings in which:

FIG. 1 is a flow chart illustrating process steps in a computer implemented method for performing deformable image registration on first and second volumetric medical images;

FIGS. 2a to 2d and 3 show flow charts illustrating further examples of methods and for performing deformable image registration;

FIG. 4 is a block diagram illustrating functional modules in an example registration node;

FIG. 5 shows a framework for implementing an example of the methods of FIG. 1, 2a to 2d, or 3;

FIG. 6 shows feature extraction according to an example;

FIG. 7 illustrates functional units in a prediction module;

FIG. 8 illustrates model architecture of a prediction network;

FIG. 9 illustrates functional units in a correction module;

FIG. 10 illustrates quantitative results of comparative experiments;

FIGS. 11, 12 and 13 illustrate qualitative results of comparative experiments;

FIG. 14 shows Table 1 illustrating quantitative results of comparative experiments;

FIG. 15 shows Table 2 illustrating quantitative results of comparative experiments;

FIG. 16 shows Table 3 illustrating the effect of a correction module in experimental results; and

FIG. 17 shows Table 4 illustrating the effect of different numbers of scales in experimental results.

### DETAILED DESCRIPTION

As discussed above, large scale deformation is a particular challenge for deformable image registration. One way to address large deformations is to consider a multi-scale problem setting, in which longer-range dependencies can be model on lower scales. LapIRN (Mok and Chung, 2020) is such a multi-scale approach for medical image registration and uses a pyramidal similarity loss. Three identical registration networks are trained progressively on pyramidal images with different scales. This multi-scale design has shown success in optical flow estimation, which is a 2-dimensional variant of medical image registration in natural images. In learning-based optical flow estimation, it has been shown that a multi-scale pyramidal feature with a single neural network can bring more benefit than pyramidal images in optical flow estimation (Sun et al., 2018). While multi-scale modelling for registration is beneficial, it has three important weaknesses. First, a blur artifacts in the predicted deformation vector field caused by bilinear/trilinear interpolation (Luo et al., 2021) lead to accumulated error in the final prediction. Second, the integration error for diffeomorphic registration is accumulated through scales. Third, the multi-scale estimation leads to the “small objects move fast” problem, in which small objects cannot be seen in lower resolution estimation and cannot be recovered in higher resolution.

Aspects of the present disclosure propose a pyramidal coarse-to-fine Prediction and Correction approach for deformable registration. The proposed method uses a pyramidal feature, for example extracted by a neural network, such that the model is able to capture high-level semantic long-range dependencies on a coarse scale. The method then starts with a, for example, zero-initialized deformation vector field in the lowest scale and predicts residual flows in each scale, in some examples using a prediction network. The deformation vector field may be progressively upsampled for example by trilinear interpolation, and added to the residual flow in each scale. To reduce the upsampling error in the residual flow prediction, some examples of the present disclosure may use a transposed convolution to predict the deformation vector field in higher resolution. In addition, in each scale, a one-step correction is performed, for example by a correction network. The final prediction in each scale thus a combination of the outputs of the prediction and correction steps. Example implementations of the method accumulate the deformation vector field from coarse to fine and consistently refine the vector field. The two steps of prediction and correction can balance the requirements of small deformation and large deformation image registration. In some examples, as the intermediate-level predictions lack details for the full resolution, the intermediate-level predictions may be constrained by a distillation loss (Luo et al., 2021) that takes the final output of the method (at full resolution) as the target for intermediate-level predictions. As the final prediction in the full resolution has the richest low-level contextual details of deformation, this provides self-guided supervision for the lower-scale predictions.

As discussed in greater detail below, the pyramidal prediction-correction framework of the present disclosure can allow for diffeomorphic registration to obtain a smooth, invertible deformation vector field that preserves topology. Specifically, a diffeomorphic implementation of a method according to the present disclosure estimates the velocity field per scale rather than directly predicting the deformation vector field. This can alleviate the integration error in each scale, which avoids damage to the full-resolution prediction.

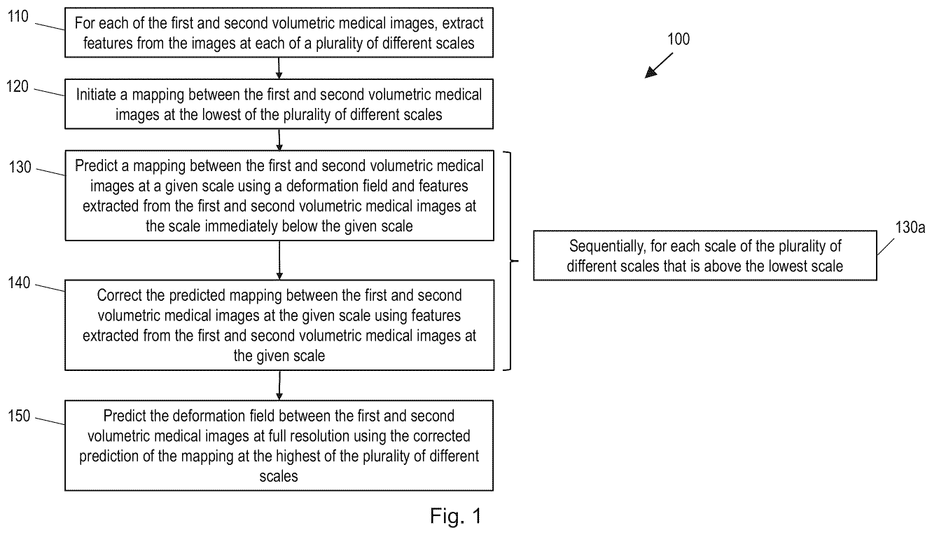

FIG. 1 is a flow chart illustrating process steps in a computer implemented method 100 for performing deformable image registration on first and second volumetric medical images. The method may be performed by a registration node, which may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below.

Referring to FIG. 1, the method 100 comprises, for each of the first and second volumetric medical images, extracting features from the images at each of a plurality of different scales in a first step 110. The method 100 then comprises, in step 120, initiating a mapping between the first and second volumetric medical images at the lowest of the plurality of different scales. As illustrated at 130a, the method 100 comprises performing steps 130 and 140 sequentially, for each scale of the plurality of different scales that is above the lowest scale. In step 130, the method comprises predicting a mapping between the first and second volumetric medical images at a given scale using a deformation field and features extracted from the first and second volumetric medical images at the scale immediately below the given scale. In step 140, the method comprises correcting the predicted mapping between the first and second volumetric medical images at the given scale using features extracted from the first and second volumetric medical images at the given scale. The method 100 further comprises, in step 150, predicting the deformation field between the first and second volumetric medical images at full resolution using the corrected prediction of the mapping at the highest of the plurality of different scales.

It will be appreciated that according to examples of the present disclosure, the prediction-correction process of steps 130 and 140 may start from the first scale up from the lowest scale, and may then work up through the scales. In some examples, the plurality of different scales may comprise a minimum of 4 different scales.

Examples of the method 100 provide improved performance in deformable image registration of volumetric medical images. This performance improvement is particularly marked for images with large scale deformation, but is also present for images with small scale deformation, as demonstrated in the experimental results presented later in the present disclosure. The first and second volumetric medical images may be obtained using any suitable imaging technology, including for example computed tomography (CT), magnetic resonance (MR), and/or ultrasound.

As discussed in greater detail below, the prediction and correction steps 130, 140 of the method 100 may be performed using Machine Learning (ML) models. For the purposes of the present disclosure, the term “ML model” encompasses within its scope the following concepts:
machine Learning algorithms, comprising processes or instructions through which data may be used in a training process to generate a model artefact for performing a given task, or for representing a real-world process or system; and
the model artefact that is created by such a training process, and which comprises the computational architecture that performs the task.

The method 100 initializes, predicts, and corrects a mapping between the first and second volumetric medical images. The mapping may in some examples comprise a deformation field between the first and second volumetric medical images or a velocity field between the first and second volumetric medical images. These concepts are discussed more fully below.

Given an image pair comprising a moving image Iₘ, and a fixed image I_{f}, image registration aims to find a transformation or deformation ϕ that minimizes the similarity function between the transformed moving image and the fixed image with regularization on the smoothness of the deformation vector field:

$${min}_{ϕ} L = {L}_{sim} ( {I}_{m} ∘ ϕ , {I}_{f} ) + ℛ ( ϕ ) \qquad Equation 1$$

where Lₛᵢₘ is the similarity loss, R is a regularization term on the deformation vector field ϕ, and Iₘ∘ϕ is the transformed moving image.

There are two common parameterizations for the deformation vector field ϕ. The first one is based on the displacement vector field:

$$ϕ ( x ) = x + u ( x ) \qquad Equation 2$$

where x is the identity transformation. With this parameterization, the displacement vector field u(x) is modeled directly. While this parameterization usually has better performance on Dice score, it does not guarantee invertibility or the preservation of the anatomical topology of organs appearing in the medical images under consideration.

The second parameterization for the deformation vector field ϕ is used for diffeomorphic deformation. In this formulation, the deformation vector field is parameterized by the velocity field v:

$$\frac{d {ϕ}^{t}}{dt} = {v}^{( t )} ( {ϕ}^{( t )} ) \qquad Equation 3$$

$${d}_{H} ( {S}_{f} , {\hat{S}}_{m} ) = max \{ {max}_{x ∈ {S}_{f}} {min}_{y ∈ {\hat{S}}_{m}} d ( x , y ) , {max}_{y ∈ {\hat{S}}_{m}} {min}_{x ∈ {S}_{f}} d ( x , y ) \}$$

such that to minimise v

$${v}^{*} = {arg max}_{v} \frac{1}{2} {\int }_{0}^{1} {‖ v ( t ) ‖}_{V}^{2} dt + {L}_{sim} ( {I}_{m} ∘ ϕ , {I}_{f} ) \qquad Equation 4$$

where v(t) ᵥ is an appropriate Sobolev norm on the velocity field (Beg et al., 2005). The final deformation vector field is obtained by integrating the velocity field (Dalca et al., 2019). The diffeomorphic formulation is smooth, invertible, and preserves topology, which can be important in a clinical setting. Prior known methods for deformable image registration do not adapt well to the diffeomorphic registration setting when applied on large deformation datasets. In contrast, examples of the present disclosure provide a diffeomorphic implementation of the methods disclosed herein which achieves improved performance, on both small- and large-scale deformation.

The following sections of the present disclosure set out additional features that may be included in the method disclosed herein, and a registration node performing the method, before providing additional detail and experimental data for a particular implementation the method.

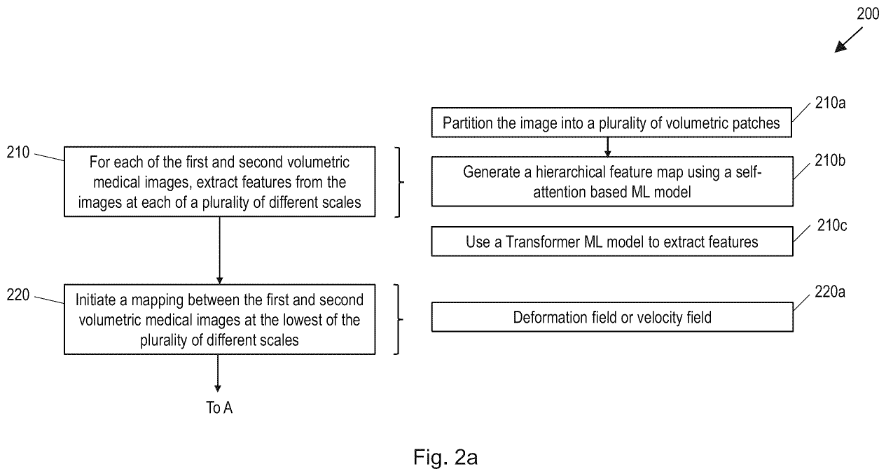

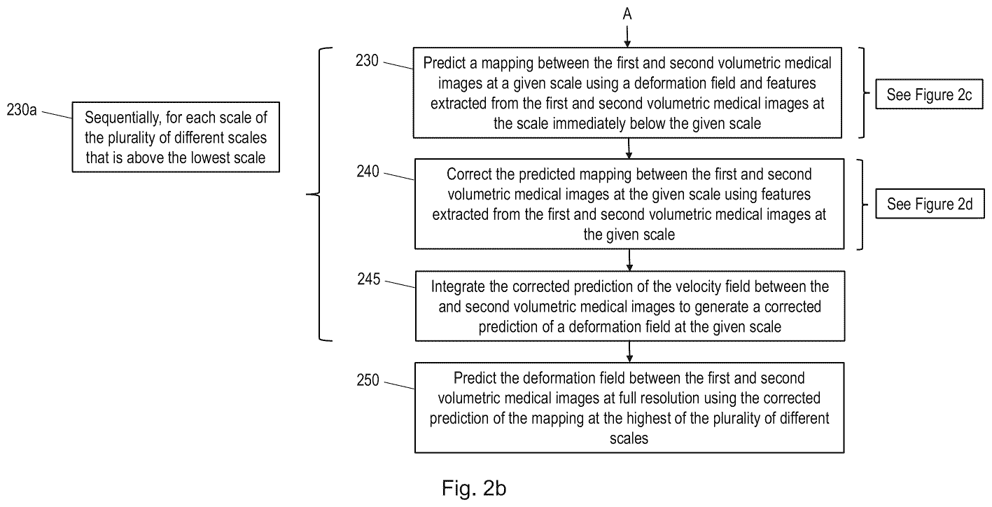

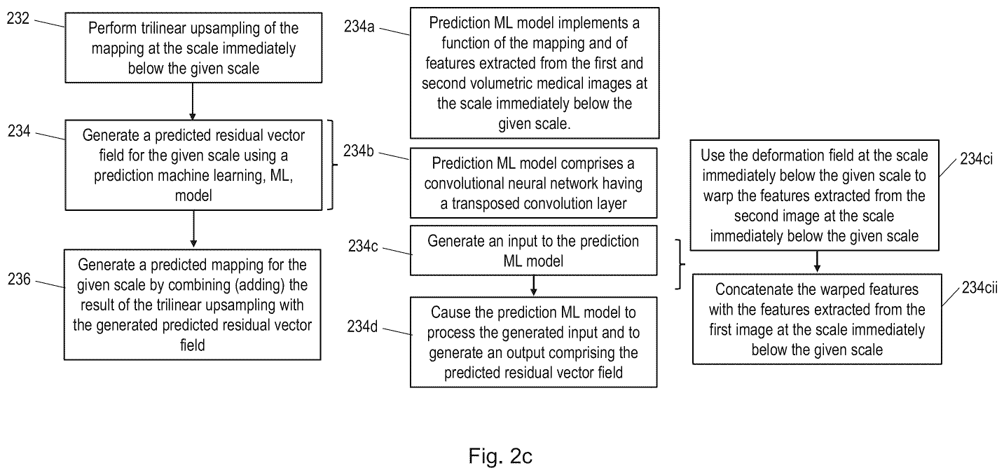

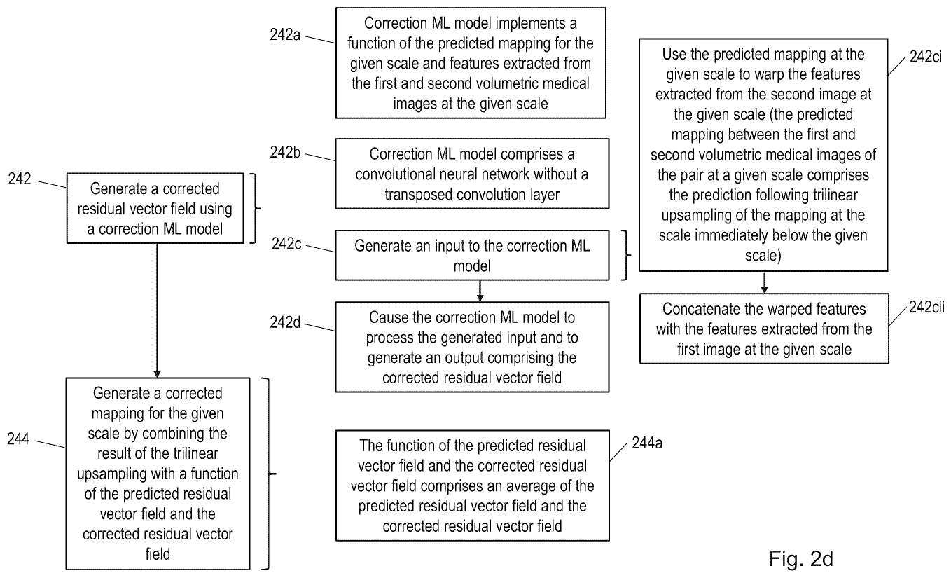

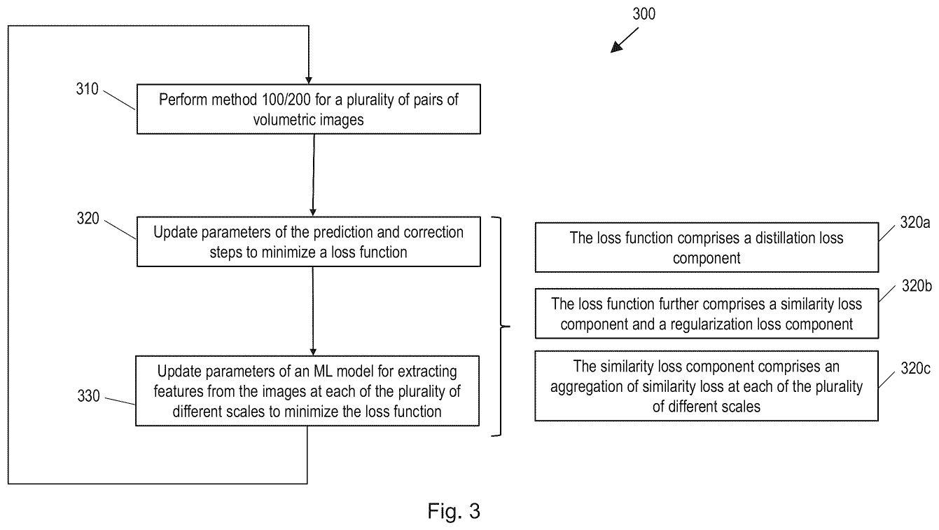

FIGS. 2a to 2d and 3 show flow charts illustrating further examples of methods 200 and 300 for performing deformable image registration. As for the method 100 discussed above, methods may be performed by a registration node, which may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below. The methods 200 and 300 illustrate examples of how the steps of the method 100 may be implemented and supplemented to provide the above discussed and additional functionality.

Referring initially to FIG. 2a, in step 210, the registration node performing the method 200 extracts features from each of the first and second volumetric medical images at each of a plurality of different scales. As illustrated in FIG. 2a, this may comprise, for an image, partitioning the image into a plurality of volumetric patches at step 210a, and generating a hierarchical feature map using a self-attention based ML model at step 210b. In some examples, extracting the features at different scales may comprise using a multiscale encoder such as U-Net, or a Swin Transformer. In some examples, extracting the features at different scales may comprise in step 210c using a Transformer ML model to extract the features. In some examples, the Transformer ML model may comprise a Swin Transformer. The number of different scales at which the features are extracted from the first and second images may vary according to particular implementations of the method 200. In some examples, a minimum of four different scales may be used. As discussed in greater detail later in the present disclosure, experimental data for a particular implementation of the methods described herein suggests that a minimum of four different scales provides a significant performance improvement over prior techniques for deformable image registration.

In step 220, the registration node initiates a mapping between the first and second volumetric medical images at the lowest of the plurality of different scales. Thus if, for example, the plurality of scales comprise ½, ¼, ⅛ and 1/16, the registration node will initiate a mapping between the first and second volumetric images at the scale of 1/16. In some examples, the registration node may initiate the mapping to zero. As discussed above and illustrated at 220a, the mapping may comprise a deformation field between the first and second volumetric medical images or, in a diffeomorphic implementation, a velocity field between the first and second volumetric medical images.

Referring now to FIG. 2b, the registration node then performs steps 230 and 240 of the method sequentially, for each scale of the plurality of different scales that is above the lowest scale, as illustrated at step 230a. The registration node therefore starts from the first scale up from the lowest scale (⅛ in the above discussed example scales), and then works up through the scales, performing the prediction step 230 and the correction step 240. In a diffeomorphic implementation of the method 200, the registration node may additionally perform the step 245 for each scale, as discussed in further detail below.

In step 230, the registration node predicts a mapping between the first and second volumetric medical images at a given scale using a deformation field and features extracted from the first and second volumetric medical images at the scale immediately below the given scale. Sub steps that may be performed by the registration node in order to complete the prediction step 230 are illustrated in FIG. 2c.

Referring now to FIG. 2c, in order to predict the mapping in the given scale, the registration node may first, in step 232, perform trilinear upsampling of the mapping at the scale immediately below the given scale. The registration node may then, at step 234, generate a predicted residual vector field for the given scale using a prediction machine learning (ML) model, before then generating a predicted mapping for the given scale by combining the result of the trilinear upsampling with the generated predicted residual vector field in step 236. In some examples, this combination may be achieved by adding the result of the trilinear upsampling to the predicted residual vector field.

As illustrated at 234a, the prediction ML model that is used to generate a predicted residual vector field for the given scale may implement a function of:
the mapping at the scale immediately below the given scale, and
features extracted from the first and second volumetric medical images at the scale immediately below the given scale.

In some examples, as illustrated at 234b, the prediction ML model may comprise a convolutional neural network having a transposed convolution layer. The transposed convolution layer may be a learnable upsampling operation, and in some examples the stride of the transposed convolution may be 2, with the remaining layers having a kernel size of 3 and a stride of 1. In one example, the prediction ML model may comprise four convolution layers and one transposed convolution layer.

In order to generate the predicted residual vector field using the prediction ML model, the registration node may first generate an input to the prediction ML model at step 234c, before causing the prediction ML model to process the generated input and to generate an output comprising the predicted residual vector field at step 234d. Generating the input at step 234c may initially comprise using the deformation field at the scale immediately below the given scale to warp the features extracted from the second image at the scale immediately below the given scale in step 234ci, and then concatenating the warped features with the features extracted from the first image at the scale immediately below the given scale in step 234cii. Thus, continuing the example of four scales, if the mapping has been initialized at the lowest scale ( 1/16), then the first time the prediction step 230 is carried out, for a given scale of ⅛, generating the input for the prediction ML model would comprise using the mapping at 1/16 scale to warp the features extracted from the second image at 1/16 scale, and then concatenating the warped features with the features extracted from the first image at 1/16 scale. The concatenation operation to combine two features for predicting the deformation vector field offers advantages in terms of memory requirements. However, in some examples, attention modules may offer additional advantages to exploit the spatial correlation between two features in an efficient and comprehensive manner.

Referring again to FIG. 2b, after the step 230 of predicting a mapping between the first and second volumetric medical images for a given scale, the registration node then performs the step 240 of correcting the predicted mapping between the first and second volumetric medical images at the given scale using features extracted from the first and second volumetric medical images at the given scale. Sub steps that may be performed by the registration node in order to complete the correction step 240 are illustrated in FIG. 2d.

Referring now to FIG. 2d, in order to predict the mapping in the given scale, the registration node may first, in step 242, generate a corrected residual vector field using a correction ML model. As illustrated at 242a, the correction ML model may implement a function of:
the predicted mapping for the given scale, and
features extracted from the first and second volumetric medical images at the given scale.

As illustrated at 242b, the correction ML model may comprise a convolutional neural network without a transposed convolution layer. Further details of an example correction ML model can be found later in the present disclosure.

As illustrated in FIG. 2d, generating the corrected residual vector field using the correction ML model may comprise generating an input to the correction ML model in step 242c, and causing the correction ML model to process the generated input and to generate an output comprising the corrected residual vector field in step 242d.

Generating the input to the correction ML model may comprise using the predicted mapping at the given scale to warp the features extracted from the second image at the given scale as illustrated at 242ci, and concatenating the warped features with the features extracted from the first image at the given scale at 242cii. As discussed above, the concatenation operation to combine two features offers advantages in terms of memory requirements. However, in some examples, attention modules may offer additional advantages to exploit the spatial correlation between two features in an efficient and comprehensive manner.

As illustrated in step 242ci, the predicted mapping between the first and second volumetric medical images of the pair at the given scale may comprise the prediction following trilinear upsampling of the mapping at the scale immediately below the given scale. In the case of a mapping comprising the deformation field, the predicted mapping at the given scale is a deformation field and so can be used directly to warp features in step 242ci. In the case of a mapping comprising a velocity field, i.e., a diffeomorphic implementation of the method, an additional step may be performed to integrate the predicted velocity field. In such examples, using the predicted mapping at the given scale to warp the features extracted from the second image at the given scale may comprise:
integrating the predicted velocity field at the given scale to obtain a predicted deformation field at the given scale, and
using the predicted deformation field at the given scale to warp the features extracted from the second image at the given scale.

In some examples, a scaling and squaring approach may be used to integrate the predicted velocity field.

Following step 242 of generating a corrected residual vector field using a correction ML model, the registration node may then generate a corrected mapping for the given scale by combining the result of the trilinear upsampling with a function of the predicted residual vector field and the corrected residual vector field in step 244. As illustrated at 244a, the function of the predicted residual vector field and the corrected residual vector field may comprise an average of the predicted residual vector field and the corrected residual vector field, and the combination may comprise an addition operation.

Referring again to FIG. 2b, and as discussed above, in a diffeomorphic implementation of the present method, the registration node may perform an additional step 245 for each of the scales. In a diffeomorphic implementation, the mapping between the first and second volumetric medical images comprises a velocity field between the first and second volumetric medical images. In step 245, the registration node may, for each scale of the plurality of different scales, integrate the corrected prediction of the velocity field between the first and second volumetric medical images to generate a corrected prediction of a deformation field at the given scale. This predicted deformation field may then be used for predicting the velocity field at the next scale up, as discussed above and illustrated in FIGS. 2b and 2c. In some examples, a scaling and squaring approach may be used to integrate the corrected velocity field.

Following execution of steps 230, 240, and in some examples 245, for each scale of the plurality of different scales that is above the lowest scale, the registration node then predicts the deformation field between the first and second volumetric medical images at full resolution using the corrected prediction of the mapping at the highest of the plurality of different scales.

FIG. 3 illustrates one example of how the methods 100, 200 may be trained. In some examples, during a training period, the registration node may, in step 310, repeat the steps of the method 100 or 200 for a plurality of pairs of first and second volumetric medical images. In step 320 the registration node may update parameters of the prediction and correction steps to minimize a loss function. The parameters of the prediction and correction steps that are updated may comprise the trainable parameters of the prediction and correction ML models. In step 330, the registration node may additionally update parameters of an ML model for extracting features from the images at each of the plurality of different scales. As discussed above, such an ML model may comprise a Swin Transformer, and the registration node may update trainable parameters of the Swin Transformer such that feature extraction also minimizes the loss function.

As illustrated at 320a, the loss function may comprise a distillation loss component, and the distillation loss component may comprise a loss between the predicted deformation field between the first and second volumetric medical images of a pair at full resolution, and the predicted deformation field between the first and second volumetric medical images of the pair at a given scale. As illustrated at 320b, the loss function may further comprise a similarity loss component and a regularization loss component. The similarity loss component may comprise an aggregation of similarity loss at each of the plurality of different scales, as illustrated at 320c. Examples of distillation, similarity and regularization loss components are discussed in further detail below.

Example methods according to the present disclosure achieve deformable image registration that offers both speed and accuracy. The methods described above offer the speed advantages associated with ML solutions when compared with classical procedures. In addition, the methods proposed herein offer improved accuracy, particularly for large deformations, as is demonstrated in the experimental data presented below. This combination of speed and accuracy can support greater speed in both the planning and delivery of radiotherapy treatment.

The speed and accuracy afforded by methods of the present disclosure can support real-time or near real-time scenarios and applications for deformable image registration. The technical benefits of this provision include reduced radiotherapy treatment plan creation time, and may result in many additional medical treatment benefits (including improved accuracy of radiotherapy treatment, reduced exposure to unintended radiation, reduced treatment duration, etc.). The methods presented herein may be applicable to a variety of medical treatment and diagnostic settings or radiotherapy treatment equipment and devices.

In one particular use case for methods of the present disclosure, a dose from a previous treatment session can be deformed or modified in light of the predicted deformation field between the first and second volumetric images at full resolution. By determining the accurate mapping of voxels from one image to another, a determination can be made as to the amount of dose delivered to a particular target depicted in the images and/or the amount of movement of the target between the times at which the images were taken. Based on the amount of delivered dose and/or movement of the target, the dose can be deformed. The output of the methods disclosed herein may thus be used in the creation or adaptation of a radiotherapy treatment plan.

Examples of the present disclosure also propose a computer implemented method for adaptation of a reference radiotherapy treatment plan, wherein the reference radiotherapy treatment plan is associated with a first volumetric medical image of a patient. The method comprises acquiring a second volumetric medial image of a patient, performing deformable image registration of the first and second volumetric medical images using a method according to any one or more of the examples described herein, and using the predicted deformation field between the first and second volumetric medical images at full resolution to adapt the reference radiotherapy treatment plan.

Examples of the present disclosure also propose a planning node for adapting a reference radiotherapy treatment plan, wherein the reference radiotherapy treatment plan is associated with a first volumetric medical image of a patient. The planning node comprises processing circuitry configured to cause the planning node execute the above discussed method.

Examples of the present disclosure also propose a radiotherapy treatment apparatus comprising a planning node as set out above.

As discussed above, the methods 100, 200 and 300 may be performed by a registration node, and the present disclosure provides a registration node that is adapted to perform any or all of the steps of the above discussed methods. The registration node may comprise a physical or virtual node, and may be implemented in a computer system, treatment apparatus, computing device, or server apparatus, and/or may be implemented in a virtualized environment, for example in a cloud, edge cloud, or fog deployment. Examples of a virtual node may include a piece of software or computer program, a code fragment operable to implement a computer program, a virtualised function, or any other logical entity. The registration node may encompass multiple logical entities, as discussed in greater detail below.

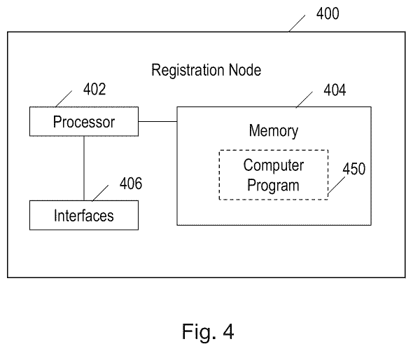

FIG. 4 is a block diagram illustrating an example registration node 400 which may implement the method 100, 200 and/or 300, as illustrated in FIGS. 1, 2a to 2d and 3, according to examples of the present disclosure, for example on receipt of suitable instructions from a computer program 450. Referring to FIG. 4 the registration node 400 comprises a processor or processing circuitry 402, and may comprise a memory 404 and interfaces 406. The processing circuitry 402 is operable to perform some or all of the steps of the method 100, 200 and/or 300 as discussed above with reference to FIGS. 1, 2a to 2d and 3. The memory 404 may contain instructions executable by the processing circuitry 402 such that the registration node 400 is operable to perform some or all of the steps of the method 100, 200 and/or 300, as illustrated in FIGS. 1, 2a to 2d and 3. The instructions may also include instructions for executing one or more telecommunications and/or data communications protocols. The instructions may be stored in the form of the computer program 450. In some examples, the processor or processing circuitry 402 may include one or more microprocessors or microcontrollers, as well as other digital hardware, which may include digital signal processors (DSPs), special-purpose digital logic, etc. The processor or processing circuitry 402 may be implemented by any type of integrated circuit, such as an Application Specific Integrated Circuit (ASIC), Field Programmable Gate Array (FPGA) etc. The memory 404 may include one or several types of memory suitable for the processor, such as read-only memory (ROM), random-access memory, cache memory, flash memory devices, optical storage devices, solid state disk, hard disk drive, etc.

In some examples as discussed above, the registration node may be incorporated into treatment apparatus, and examples of the present disclosure also provide a treatment apparatus, such as a radiotherapy treatment apparatus, comprising either or both of a registration node as discussed above and/or a planning node operable to implement a method for adapting a radiotherapy treatment plan, also as discussed above.

FIGS. 1 to 3 discussed above provide an overview of methods which may be performed according to different examples of the present disclosure. These methods may be performed by a registration node, as illustrated in FIG. 4.

As will be appreciated from the above discussion, example methods disclosed herein propose a pyramidal prediction correction framework for deformable image registration. In some examples, a predicted residual vector field is progressively added to an initial deformation vector field, and this is consistently refined by a correction network on different scales. The methods disclosed herein may be extended to allow for diffeomorphic registration without performance degradation, for example using a scaling and squaring approach to integrate predicted or corrected velocity fields. In training the proposed methods, a distillation loss may be used for self-guided training of pyramidal medical image registration. As is demonstrated below, experiments on challenging datasets, including large deformations, show significant improvements compared to the state-of-the-art.

There now follows a detailed discussion of how different process steps illustrated in FIGS. 1 to 3 and discussed above may be implemented, as well as presentation of experimental results for the example implementation. The functionality and implementation detail described below is discussed with reference to the modules of FIG. 4 performing examples of the methods 100, 200 and/or 300, substantially as described above.

### Implementation Architecture

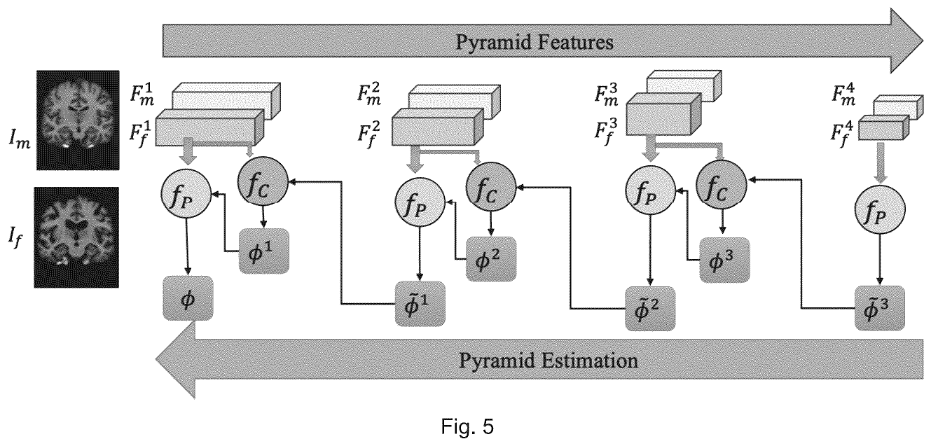

An architecture implementing examples of the methods 100, 200 and/or 300 may be divided into three components: a feature extractor, a predictor, and a corrector, and an overall framework is shown in FIG. 5. With reference to FIG. 5, features of both moving and fixed images are extracted in a pyramidal way. F¹, F², F³, F⁴ are features at scales ½, ¼, ⅛, 1/16, respectively. f_{P} represents the prediction module. f_{C} is a correction module. ϕ is the predicted deformation vector field. The deformation vector field is first updated by the prediction module and then refined by the correction module

In the following discussion, it is assumed that the first image of the pair of volumetric medical images in a fixed image, and the second image of the pair is a moving image. A feature extractor is first used to obtain features from the moving and fixed images in different scales. In each scale, a predictor is then used to estimate the residual vector field to update the deformation (or velocity) field and a corrector module to refine the deformation (or velocity) field. The deformation (or velocity) field is progressively updated through different scale predictions. The implementation discussed and evaluated below is referred to as PC-Reg, and PC-Reg-diff, for the diffeomorphic implementation.

### Feature Extraction (Steps 110, 210 of Methods 100, 200)

The present example implementation uses a variant of Swin transformer Liu et al. (2021) as feature extractor. Given an image pair, Iₘ and I_{f}, pyramidal features Fₘ^{l} and F_{f}^{l}, I=1, . . . , 4, are first extracted, where I denotes the scale level of features. In contrast to the original Swin transformer and that used in Chen et al. (2022), the 3D input volumes are first partitioned into different patches by two convolution residual blocks with feature dimensions 32 and 64, respectively. Each block consists of two 3D convolution layers with instance normalization layers. The partitioned patch has the size of 4×4×4. Three stages of patch partition and Swin Transformer blocks are then used. Patch partition is for downsampling the features, while each Swin Transformer block consists of one multi-head attention (MSA) and one shifted window-based self-attention (SW-MSA). There are 2, 2, and 12 Swin Transformer blocks in each stage, respectively. Features are extracted for two images separately to obtain the feature maps at ½, ¼, ⅛, and 1/16 scales.

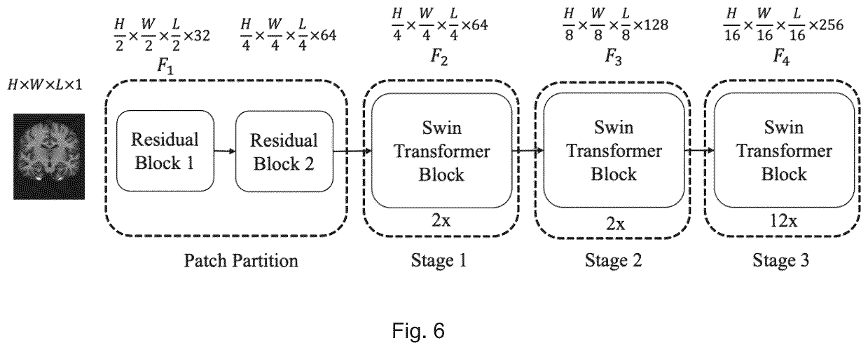

FIG. 6 illustrates feature extraction by Swin Transformer blocks. Two residual blocks are used for partitioning the image into patches, followed by three stages of patch merging and Swin Transformer blocks. There are 2, 2, and 12 Transformer blocks in each stage, respectively. Between every two stages, features are downsampled. As shown in FIG. 6, the input images Iₘ and I_{f} have the size of H×W×L, and the extracted features F^{l} have the size of

$$\frac{H}{{2}^{l}} \times \frac{H}{{2}^{l}} \times \frac{H}{{2}^{l}} \times ( 32 * {2}^{l} ) , l = 1 , … , 4.$$

H, W, L are the height, width, and length of the 3D image, and isotropic voxel size is assumed. A feature map at scale I has double the spatial size as a feature map at scale level I+1.

Residual Vector Field Prediction (steps 120, 130, 220, 230, 232-236 of methods 100 and 200)

The following discussion relates to prediction of deformation fields. A diffeomorphic implementation, and prediction of vector fields, discussed separately below.

After extracting the features of moving and fixed images, coarse-to-fine deformation vector fields are predicted. Pyramidal optical flow methods commonly use bilinear interpolation to upsample the estimated flow field between every two scales. The upsampled flow field is then used to warp the moving image feature for predicting a new flow field in the current scale. This works well in 2D natural images (Sun et al., 2018). However, 3D medical images have more complex structures and may have large deformations, leading to large cumulative errors when upsampling, as well as during the integration of the velocity fields in the diffeomorphic setting. To alleviate this issue, examples of the present disclosure use the above discussed the prediction-correction model. The present section considers the prediction module.

Starting from a zero-initialized deformation vector field ϕ^{l+1} at the lowest scale (I+1=4), ϕ is progressively upsampled, and a prediction network fₚ^{l} is used to predict a residual vector field to refine the upsampled vector field at each scale. Specifically, the prediction network relies on a transposed convolution layer to predict a residual vector field in higher scales with learnable parameters.

$${\tilde{ϕ}}^{l} = Up ( {ϕ}^{l + 1} ) + {f}_{P} ( {ϕ}^{l + 1} , {F}_{m}^{l + 1} , {F}_{f}^{l + 1} ) \qquad Equation 5$$

$${\tilde{ϕ}}^{l} = {ϕ}_{0}^{l} + {R}_{0}^{l} \qquad Equation 6$$

where ϕ₀^{l}=Up(ϕ^{l+1}), and R₀^{l}=fₚ(ϕ^{l+1}, Fₘ¹⁺¹, F_{f}¹⁺¹). Up is the upsampling operation.

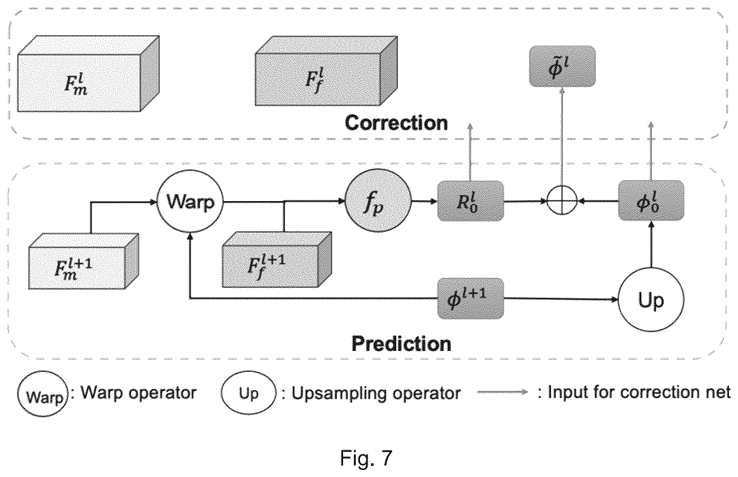

The inputs for the prediction module are the deformation vector field at scale I+1, ϕ^{l+1} and the moving and fixed image features (Fₘ¹⁺¹ and F_{f}¹⁺¹). FIG. 7 illustrates functional units of the prediction module. First, the deformation field from the last scale ϕ^{l+1} is used to warp the feature Fₘ¹⁺¹. Then the prediction network f_{P} estimates a residual vector field R₀^{l}

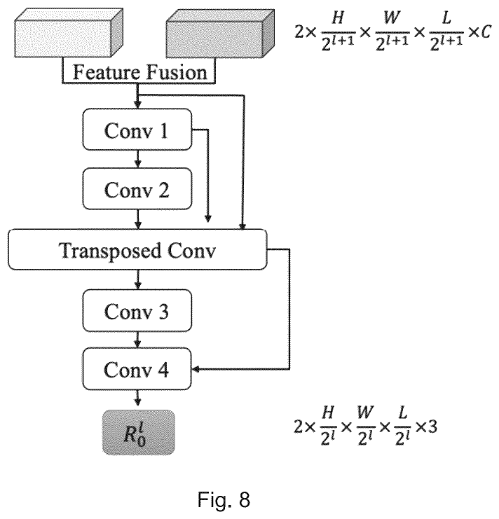

As shown in FIG. 7, the deformation vector field ϕ^{l+1} is first used to warp Fₘ¹⁺¹, then, Fₘ¹⁺¹∘ϕ^{l+1} is concatenated with F_{f}^{l+1} as the input of prediction network f_{P}. The architecture of the prediction network f_{P} is illustrated in FIG. 8. The prediction network consists of four convolution layers and one transposed convolution layer. The transposed convolution is a learnable upsampling operation. By using the stride of transposed convolution as 2, the residual vector field is predicted in higher resolution. For the rest of the convolution layers, the kernel size is 3 with stride 1. In order to allow for better gradient flows during training, the DenseNet Huang et al. (2017) design is adopted, where each layer is densely connected to other layers.

Residual Vector Field Correction (steps 140, 240, 242-244 of methods 100 and 200) FIG. 7 illustrates how to obtain \tilde{ϕ}^{l} from the features and deformation vector field at scale I+1. The input for the correction step is the predicted deformation vector field \tilde{ϕ}^{l}, the initial deformation vector field ϕ₀^{l} at scale I, and the residual vector field R₀^{l}=Fₚ(ϕ^{l+1},Fₘ¹⁺¹, F_{f}¹⁺¹). The correction step may be envisaged as inspired by the predictor-corrector method (Butcher, 2016) in numerical methods for differential equations. For a differential equation

$${y}^{′} = f ( t , y ) \qquad Equation 7$$

with initial condition y(t₀)=y₀ and step size h, the equation can be solved numerically by:

$${\tilde{y}}_{i + 1} = {y}_{i} + hf ( {t}_{i} , {y}_{i} ) \qquad Equation 8$$

$${y}_{i + 1} = {y}_{i} + \frac{1}{2} h ( f ( {t}_{i} , {y}_{i} ) + f ( {t}_{i + 1} , {\tilde{y}}_{i + 1} ) ) \qquad Equation 9$$

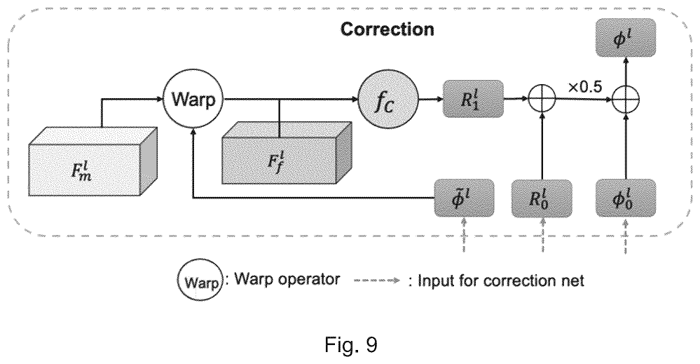

The above described prediction step may be thought of as analogous to Eq.8 with step size h=1. Examples of the present disclosure propose a correction network f_{C} to do one step further correction on the deformation vector field that. A correction module, comprising a correction network f_{C} is presented in FIG. 9, with \tilde{ϕ}^{l}, R₀^{l}, and ϕ₀^{l} being outputs from the prediction module. Eq. 6 is rewritten, and the prediction correction step may be presented as:

$${\tilde{ϕ}}^{l} = {ϕ}_{0}^{l} + {R}_{0}^{l} \qquad Equation 10$$

$${ϕ}^{l} = {ϕ}_{0}^{l} + \frac{1}{2} ( {R}_{0}^{l} + {R}_{1}^{l} ) \qquad Equation 11$$

$${R}_{1}^{l} = {f}_{C} ( {\tilde{ϕ}}^{l} , {F}_{m}^{l} , {F}_{f}^{l} ) \qquad Equation 12$$

An intuitive interpretation is that the features from the current scale are used to enrich the details and correct the predicted deformation vector field \tilde{ϕ}^{l}. As \tilde{ϕ}^{l} is predicted based on the lower resolution features, Fₘ^{l} and F_{f}^{l}, it has a better perception of the global information but may lack detailed local information. Hence, the correction step (Eq. 11) comprises \tilde{ϕ}^{l} with the higher resolution features for updating the deformation vector field.

This correction step is for alleviating the error caused by the upsampling or the velocity field numerical integration.

The correction network f_{C}_{_{f}} is implemented as a four-layer convolutional network without transposed convolution layer and with dense skip connections. Leaky ReLu activations are used as well as instance normalization between each layer. The input of f_{C} is the concatenation of the transformed moving feature by \tilde{ϕ}^{l}, Fₘ^{l} ∘\tilde{ϕ}^{l} and the feature of fixed image F_{f}^{l}. The output is a residual vector field R₁^{l}.

Overall, the method performs coarse-to-fine deformation vector field prediction and correction, with the predicted deformation vector field being progressively refined. The final output is estimated by one-step prediction without correction as features have not been extracted in full resolution. In the experiments detailed below, the effectiveness of the proposed correction module is evaluated through an ablation study.

### Diffeomorphic Registration (Step 245 and 242ci of Method 200)

As discussed above, the prediction-correction registration network can be used to support diffeomorphic registration, and thus predict deformation vector fields that are smooth and invertible. Instead of the deformation vector fields, the diffeomorphic implementation of the methods disclosed herein (PC-Reg-diff) predicts the velocity fields in each scale. Starting with a zero-initialized velocity field, PC-Reg-diff uses prediction and correction modules to update and refine the velocity field at each scale iteratively, accumulating the velocity fields. As the prediction and correction modules are based on the transformed feature of the moving image, the scaling-and-squaring approach (Dalca et al., 2019) is applied to obtain the deformation vector field in each scale. The obtained deformation vector fields are then used to transform the moving image.

In diffeomorphic parameterization, the velocity field v in Eq. 3 is integrated to obtain the deformation vector field. It is proposed to follow the previous works (Arsigny et al., 2006; Dalca et al., 2019; Mok and Chung, 2020) by using a computationally efficient scaling-and-squaring approach for integration. The scaling-and-squaring approach considers the velocity field as stationary, and integrates the velocity field from time 0 to 1 with time steps T. The deformation vector field is represented as a Lie algebra member that is exponentiated to generate a time 1 deformationϕ¹=exp(ν). The scaling-and-squaring recurrence starts with

$${ϕ}^{( 1 / {2}^{T} )} = p + \frac{v ( p )}{{2}^{T}} \qquad Equation 13$$

where T is the number of steps for integration, and p denotes the spatial locations. In the present example T=7. The ϕ⁽¹⁾ can be obtained using the recurrence:

$${ϕ}^{( 1 / {2}^{t - 1} )} = {ϕ}^{( 1 / {2}^{t} )} ∘ {ϕ}^{( 1 / {2}^{t} )} \qquad Equation 14$$

$$Thus ,$$

$${ϕ}^{( 1 )} = {ϕ}^{( 1 / 2 )} ∘ {ϕ}^{( 1 / 2 )} .$$

### Loss (Training Method 300 Including Steps 320 and 330)

The overall loss used in the presently described implementation consists of three parts: a similarity loss Lₛᵢₘ, a regularization loss L_{R}, and a distillation loss L_{DT}. The overall unsupervised loss is defined by:

$$L = {L}_{sim} + {λ}_{R} {L}_{ℛ} + {λ}_{D T} {L}_{D T} \qquad Equation 15$$

where λ_{R} and λ_{DT} are hyperparameters for weighing different parts.

Similarity loss: Following (Balakrishnan et al., 2019; Chen et al., 2022), the local normalized cross-correlation (LNCC) loss is used as the basis for the similarity loss

$${L}_{NCC} ( {I}_{m} , {I}_{f} , ϕ ) = {\sum }_{p ∈ ω} \frac{{\sum }_{{p}_{i}} ( {I}_{f} ( p ) - {\overline{I}}_{f} ( p ) ) {( [ {I}_{m} ∘ ϕ ] ( {p}_{i} ) - [ {\overline{I}}_{m} ∘ ϕ ] ( p ) )}^{2}}{\begin{matrix}( {\sum }_{{p}_{i}} {( {I}_{f} ( {p}_{i} ) - {\overline{I}}_{f} ( p ) )}^{2} ) ( {\sum }_{{p}_{i}} ( [ {I}_{m} ∘ ϕ ] ( {p}_{i} ) - \\ {[ {\overline{I}}_{m} ∘ ϕ ] ( p ) )}^{2} )\end{matrix}} \qquad Equation 16$$

where p is the voxel coordinates, ω is the image domain, and Ī denotes the mean voxels within the window centered at voxel p. L_{NCC} measures the local similarity within sliding windows. To compute the losses for all intermediate outputs from different scales, a multi-scale similarity loss Lₛᵢₘ is used that is similar to LapIRN (Mok and Chung, 2020):

$${L}_{sim} = {\sum }_{i} \frac{1}{{2}^{i - 1}} {L}_{NCC} ( Pool ( {I}_{m} ) , Pool ( {I}_{f} ) , {ϕ}^{i} ) \qquad Equation 17$$

where Pool is the average pooling operation. In contrast to the multi-scale similarity loss in LapIRN (Mok and Chung, 2020), where the predicted deformation vector field in different scales is used separately in different stages, in the present implementation, multi-scale predictions are aggregated simultaneously and the model is trained in an end-to-end manner.

Regularization loss: In displacement field parameterization, training with a sole similarity loss may lead to non-smooth and unrealistic deformations. To avoid this, a regularization loss is added over the deformation vector field. The diffusion regularizer from (Balakrishnan et al., 2019) is used as smoothness regularization. As for the similarity loss, the smoothness loss is defined over deformation vector field predictions from all scales:

$${L}_{ℛ} = {\sum }_{i} \frac{1}{{2}^{i - 1}} {\sum }_{p ∈ ω} {‖ ∇ {ϕ}^{i} ( p ) ‖}^{2} \qquad Equation 18$$

Distillation Loss: In addition to the above terms, as the prediction relies on the intermediate deformation vector field estimations, it is helpful to provide correct guidance for coarse predictions during training. One way is to define the similarity loss over intermediate outputs as mentioned above. In addition to this, examples off the present disclosure introduce a distillation loss that uses the final deformation vector field output as the target to supervise the intermediate outputs. The intermediate flows are encouraged to resemble the predicted flow in full resolution. By transferring the information from full-resolution to low-resolution predictions, the intermediate predictions are able to perceive moving fast small objects thanks to feedback from the fine-grained output. This idea may be understood as being similar to knowledge distillation. With reference to (Luo et al., 2021), the distillation loss is defined by the L1 loss between the upsampled deformation vector field prediction Vᵢ at scale land the final prediction V:

$${L}_{D T} = {\sum }_{i} {α}_{i} ( ‖ ϕ - Up ( {ϕ}^{i} ) ‖ ) \qquad Equation 19$$

where αᵢ is weighting hyperparameter in scale i, and Up is the trilinear upsampling function. An ablation study has also been conducted on the distillation loss to show its effectiveness.

In some examples, experiments are conducted in a semi-supervised setting. To this end, an additional Dice loss is used together with the L_{NCC} loss as the similarity loss. The Dice loss is calculated between the warped moving segmentation mask by the estimated deformation vector field and the fixed segmentation. The Dice loss is defined by:

$${L}_{DSC} = 1 - D S C ( {S}_{f} , {S}_{m} ) \qquad Equation 20$$

$$D S C ( {S}_{f} , {S}_{m} ) = \frac{1}{K} {\sum }_{k = 1}^{K} \frac{\vert {S}_{f}^{k} ∩ ( {S}_{m}^{k} ∘ ϕ ) \vert}{\vert {S}_{f}^{k} \vert + \vert {S}_{m}^{k} ∘ ϕ \vert} \qquad Equation 21$$

where K is the number of labels, S_{f} and Sₘ are the segmentation masks of fixed and moving images. The weight of Dice loss is set as 1.

Examples of the present disclosure thus provide a pyramidal framework for deformable medical image registration, which framework is particularly suited to image sets demonstrating large deformations. Example methods according to the present disclosure use pyramidal feature extraction in combination with prediction and correction networks for deformation vector field estimation in each scale. The methods progressively predict a residual vector field to update a deformation or velocity vector field, and a correction vector field to refine predicted field through different scales. In some examples, a distillation loss is introduced to provide self-guidance using the full-resolution prediction during training. Methods according to the present disclosure can also be implemented for diffeomorphic registration to obtain a smooth and invertible deformation vector field, and providing a superior advantage on a large deformation dataset. The experimental results presented show that the methods disclosed herein perform well on OASIS and ABD50 datasets.

The methods of the present disclosure may be implemented in hardware, or as software modules running on one or more processors. The methods may also be carried out according to the instructions of a computer program, and the present disclosure also provides a computer readable medium having stored thereon a program for carrying out any of the methods described herein. A computer program embodying the disclosure may be stored on a computer readable medium, or it could, for example, be in the form of a signal such as a downloadable data signal provided from an Internet website, or it could be in any other form.

It should be noted that the above-mentioned examples illustrate rather than limit the disclosure, and that those skilled in the art will be able to design many alternative embodiments without departing from the scope of the appended claims or numbered embodiments. The word “comprising” does not exclude the presence of elements or steps other than those listed in a claim or embodiment, “a” or “an” does not exclude a plurality, and a single processor or other unit may fulfil the functions of several units recited in the claims or numbered embodiments. Any reference signs in the claims or numbered embodiments shall not be construed so as to limit their scope.

### Experimental Data

The Experiments detailed below are conducted on OASIS dataset (Marcus et al., 2007), and a well-annotated subset of AbdomenCT-1 K dataset (Ma et al., 2021).

OASIS In this study, we perform inter-patient registration experiments on 414 T1-weighted brain MRI scans from the OASIS dataset Marcus et al. (2007), which includes scans of Alzheimer patients with mild to moderate symptoms. We use the pre-processed OASIS dataset provided by the Learn2Reg challenge (Hering et al., 2022). The scans were pre-processed using the standard techniques provided by FreeSurfer Fischl (2012), including normalization and skull stripping. Each scan has a spatial size of 160×192×224 and an isotropic voxel size of 1 mm³. In addition, segmentation masks of 35 anatomical structures are available. We randomly select the dataset into 294 images for training, 20 for validation, and 100 for testing. For the testing set, we select every two consecutive images as pairs, resulting in 99 pairs.

ABD50 For CT-CT registration, we use a subset of AbdomenCT data Ma et al. (2021) as this subset provides notated segmentation masks. It has 50 cases with 12 annotated organs: liver, kidney, spleen, pancreas, esophagus, gallbladder, stomach, aorta, celiac trunk, inferior vena cava, right adrenal gland, and left adrenal gland. Due to varying spatial size, we resample each volume to 224×224×96. Moving images were resampled based on the voxel size of fixed images. We randomly select 35, 5, and 10 volumes for training, validation, and test sets, respectively. Due to the limited number of data, we combine every two volumes in the test set for a comprehensive evaluation, which gives us 90 testing pairs. The Dice score of test pairs in ABD50 is low, which means it is initially largely deformed. Hence, we use this dataset to evaluate the model performance on large deformation.

### Measurements

We evaluate the model performance by Dice score (DSC) (Dice, 1945), Hausdorff Distance and log Jacobian determinant score, which are also used in previous works (Bal-akrishnan et al., 2019; Mok and Chung, 2020) and Learn2Reg challenge (Hering et al., 2022). The final score (mean and standard deviation) is obtained by averaging all the test pairs.

DSC Due to most datasets not having ground truth correspondence (dense/sparse point-wise relationship) available, DSC (Eq. 21) is used to measure the overlap between the segmentation maps of the deformed moving images and fixed images. We provide the mean and standard deviation of the Dice score.

Hausdorff Distance Hausdorff Distance is a metric to evaluate the maximum distance of a set to the nearest point in the other set. It is defined by:

$${d}_{H} ( {S}_{f} , {\hat{S}}_{m} ) = max \{ {max}_{x ∈ {S}_{f}} {min}_{y ∈ {\hat{S}}_{m}} d ( x , y ) , {max}_{y ∈ {\hat{S}}_{m}} {min}_{x ∈ {S}_{f}} d ( x , y ) \} \qquad Equation 22$$

where Ŝₘ=Ŝₘ^{k} ∘ϕ and d is the Euclidean distance. Following the protocol of Learn2Reg challenge (Hering et al., 2022), instead of using the original Hausdorff distance, we adopt a robust variant, 95% Hausdorff distance. This measures the 95th percentile of the distances between boundaries of warped moving image segmentation and fixed image segmentation. We account for the voxel size and use the voxel size of the fixed image during evaluation.

Jacobian Determinant Similarly to other learning-based methods (Balakrishnan et al., 2019; Chen et al., 2022), we use |J_{ϕ}(p)|=|∇ϕ(p)|≤0 to evaluate the percentage of folding voxels of the deformation vector field, where p denotes the voxel location and |⋅| is the determinant.

### Baseline Methods

We compare our methods with different state-of-the-art conventional methods and learning-based models.

Conventional Methods We used the conventional method, Symmetric Normalization (SyN)(Avants et al., 2008) as our baseline. For the implementation, we use the public package, Advanced Normalization Tools (ANTs) (Avants et al., 2011). In addition, NiftyReg (Modat et al., 2010), open-source software was used as the second baseline.

Learning-based Methods We compare our model with three learning-based methods, VoxelMorph Balakrishnan et al. (2019), LapIRN Mok and Chung (2020) and TransMorph Chen et al. (2022). For all three models, we use the local normalized cross-correlation loss and the smoothness regularization loss with weights λ_{R} as 1. For semi-supervised training, we add a DSC loss for each method. We compare the model performance with and without a diffeomorphic parameterization. LapIRN is originally a diffeomorphic registration method with a displacement vector field variant, LapIRN-disp. For VoxelMorph and TransMorph, a scaling-and-squaring integration module is added on top of the prediction for the diffeomorphic registration. We denote the diffeomorphic variants as VoxelMorph-diff and TransMorph-diff.

For TransMorph, we use the default TransMorph-Large setting from (Chen et al., 2022), which has a 128 initial embedding dimension. In the ABD50 dataset, 7×7×4 window size is used to make the TransMorph compatible with the image size in the dataset. As for LapIRN, it has a three-stage training strategy on scales ½, ¼, and full resolution, respectively. To have a fair comparison, we train LapIRN with the same iterations in the third stage as other methods. In the semi-supervised setting, the DSC loss is added in the third stage with the full resolution.

### Experimental Implementation

In our experiments, we adopt Adam optimizer Kingma and Ba (2014) with a learning rate 1ₑ-4. The batch size is 1, and we empirically set λ_{R}=1, λ_{DT}=2. For the similarity loss, the window size of L_{NCC} is 9 in full resolution. We train the above described implementation of methods disclosed herein, referred to as PC-Reg and PC-Reg-diff respectively, in both unsupervised and semi-supervised manners. For the semi-supervised training, the weight for DSC loss is 1. We use three stages of Swin Transformer blocks in all our experiments. In the ABD50 dataset, we employ a window size of 7×7×4 to make the model compatible with the data. Our experiments are conducted in an A100 GPU with 40 GB memory

### Experimental Results

In this section, we present the quantitative and qualitative performance of the proposed methods, PC-Reg and PC-Reg-diff, on different datasets.

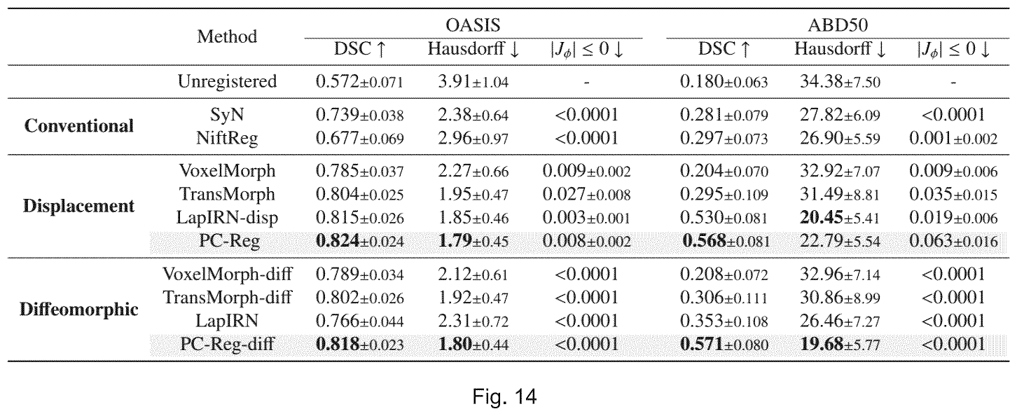

Unsupervised comparison The unsupervised results of different methods on OASIS and ABD50 are shown in Table 1, presented in FIG. 14. Conventional indicates the conventional methods, while Displacement and Diffeomorphic are learning-based methods with displacement field and diffeomorphic parameterization, respectively. All methods and their diffeomorphic variants are compared. ⬆: the higher the better, ⬇: the lower the better. We first present the results with displacement vector field parameterization. In both datasets, PC-Reg has the best performance on both the Dice score and the Hausdorff score. The experimental results show that multi-scale approaches, LapIRN-disp and PC-Reg are advantageous compared to single-scale approaches (VoxelMorph and TransMorph). Specifically, on the large displacement dataset, ABD50, PC-Reg outperforms VoxelMorph and TransMorph by large margins of up to 20 and 26 percentage points of Dice score, respectively. In ABD50, the initial mean Dice score of 12 annotated organs is around 0.18. Hence, the dataset is challenging due to the large displacements. This shows that the multi-scale design can better capture the long-range displacement. Within multi-scale approaches, PC-Reg has a better performance than LapIRN by 1% Dice score on the OASIS dataset and around 4% in the ABD50 dataset.

Considering the diffeomorphic variants, the proposed method implementation, PC-Reg-diff, outperforms all baselines. In ABD50 we notice that PC-Reg-diff improves PC-Reg around 0.3% Dice score and 3 Hausdorff score, while the counterpart multi scale approach, LapIRN, observes a decreased performance. More generally, PC-Reg-diff outperforms all other diffeomorphic methods by 20-37 percentage points, including LapIRN. We believe this is due to our correction step alleviating the integration error in multiple scales. The reason is that adding the velocity field from different scales can be regarded as the Euler method of integration in numerical methods for differential equations. By contrast, the predictor-corrector method for integration is more numerically stable. Furthermore, we conducted the paired Wilcoxon tests between PC-Reg(-diff) and other learning-based methods. The p-value is less than 0.005.

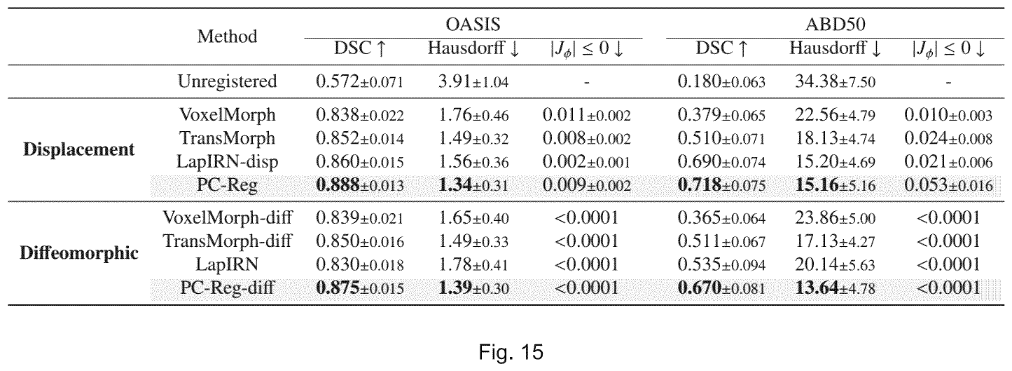

Semi-supervised comparison Since we are interested in finding out which method can attain the best accuracies over-all, we also conducted semi-supervised experiments of different methods on OASIS and ABD50, which are shown in Table 2, presented in FIG. 15. Displacement and Diffeomorphic are learning-based methods with displacement field and diffeomorphic parameterization, respectively. All methods and their diffeomorphic variants are compared. ⬆: the higher the better, ⬇: the lower the better. In the OASIS dataset, PC-Reg and PC-Reg-diff have the best Dice and Hausdorff performance, outperforming all other learning-based baselines and their diffeomorphic variants, that is LapIRN(-disp), TransMorph(-diff), and VoxelMorph(-diff), by 2%-5% in Dice. Furthermore, in the ABD50 dataset, PC-Reg-diff significantly outperforms (p-value<0.005) other methods by 10%-30% in Dice. Overall, PC-Reg-diff has a stable and relatively good performance on both OASIS (small deformation) and ABD50 (large deformation) datasets.

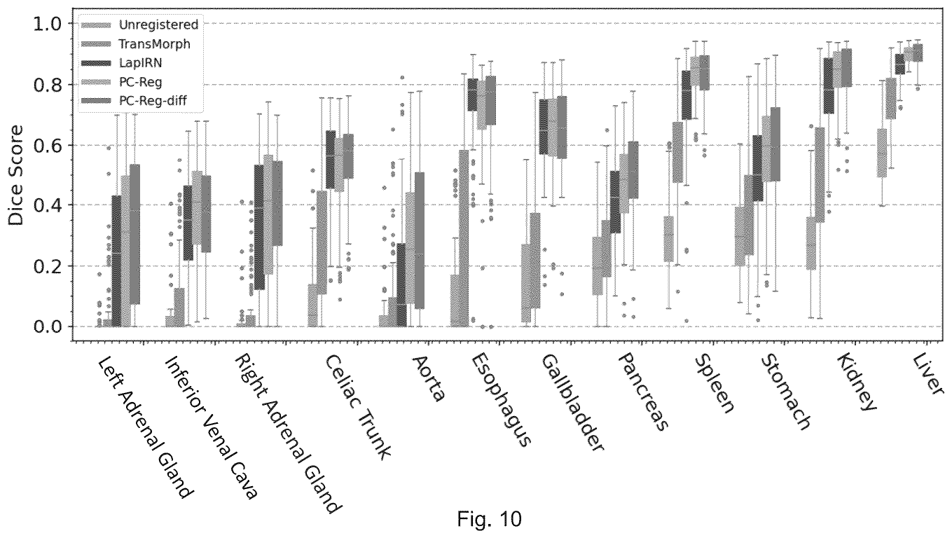

Performance on each region In order to demonstrate the strengths of the proposed methods on organs which undergo large deformations, we further present results per organ of ABD50 in FIG. 10. FIG. 10 shows quantitative results of different methods on each sub-region of ABD50 dataset. Organs are ordered by organ size. PC-Reg has a better performance in extreme cases where the organs almost do not overlap initially. Another interesting point is that large deformation often corresponds to organs with very small sizes and could be seen as “fast-small objects”. Importantly, PC-Reg and PC-Reg-diff improve accuracy with such small objects undergoing large deformations, as well as with large objects undergoing medium deformations.

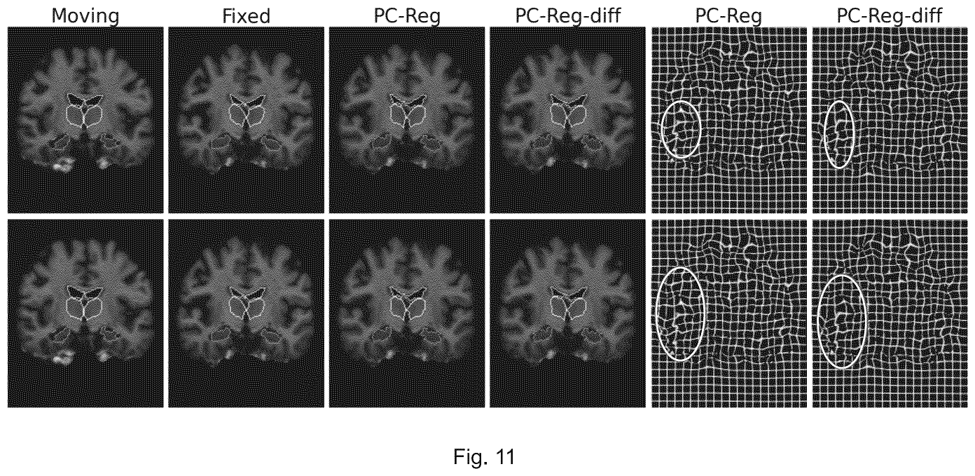

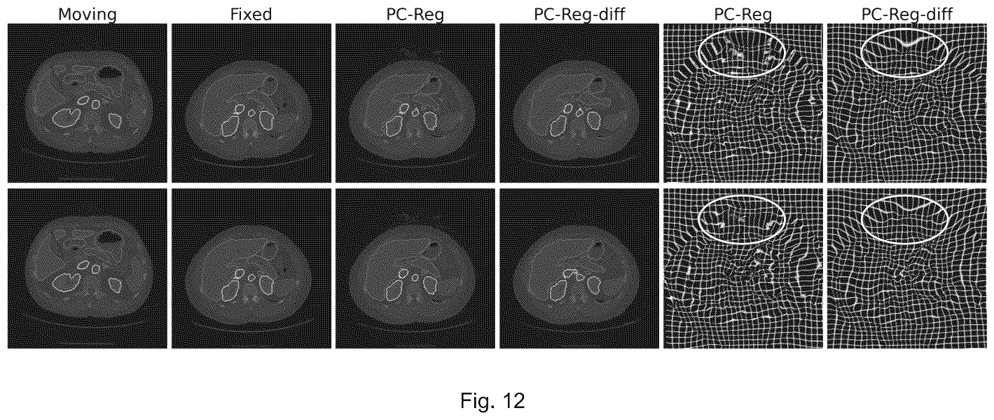

Qualitative results The qualitative results of our method on the OASIS dataset are illustrated in FIG. 11. The first row shows the unsupervised results, while the second row shows the semi-supervised results. The contours of the ventricles, third ventricle, thalami, and hippocampi are shown in the figure. The segmentation contour of four sub-regions, including the ventricles, third ventricle, thalami, and hippocampi are shown in the visualization. It can be seen that the proposed diffeomorphic method PC-Reg-diff has a smoother deformation vector field. The qualitative results of our method on the ABD50 dataset are illustrated in FIG. 12. The first row shows the unsupervised results, while the second row shows the semi-supervised results. We demonstrate the qualitative results of our methods on OASIS and ABD50 datasets. The diffeomorphic variant of our method makes the deformation vector field much smoother, especially in the ABD50 dataset.

### Ablation Study

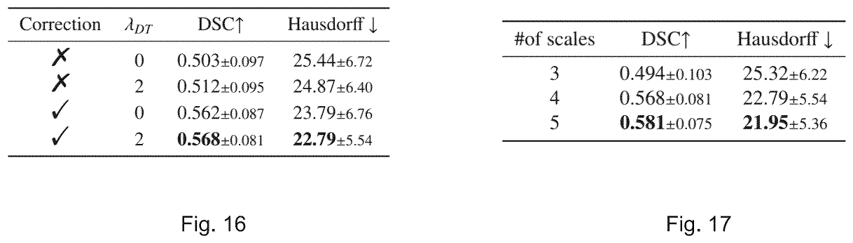

In order to evaluate the effectiveness of our proposed correction module and distillation loss, we conducted an ablation study over these two modules with displacement field parameterization. Table 3, presented in FIG. 16, shows the effect of the correction module and distillation loss on ABD50. The method with the correction module and the distillation loss achieves consistently better performance. As shown in Table 3, the correction module brings 6% improvement of the Dice score. Moreover, the distillation loss with weight 2 gives a further improvement on both Dice and Hausdorff scores with and without the correction module. Hence, the correction module is crucial for multi-scale prediction. Also, the distillation is beneficial for transferring detailed textual information from the full-resolution prediction.

In addition, we compare our model performance with respect to the number of the scales of our prediction on the ABD50 dataset in Table 4, presented in FIG. 17. Our default model uses three stages of Swin Transformer blocks and predicts the deformation in four scales, including the full resolution. Decreasing/increasing the stages of Swin Transformer blocks will result in fewer or more features, which changes the number of scales prediction. However, as our model relies on the feature extractor, fewer scales mean fewer layers/depths of our feature extractor network, which reduces the receptive field of the feature and gives fewer representative features. Hence, we can observe a decrease in the performance with three scales prediction. Similarly, with more scales, we have a deeper feature extractor and more intermediate constraints, which leads to an even better performance of around 0.581 Dice score. For all experiments, we use the same λ_{DT}=2. PC-Reg achieves the best performance with five scale predictions on the ABD50 dataset. As the number of scales that we can obtain depends on the image size, we use 4 scales in all other experiments for general usage.

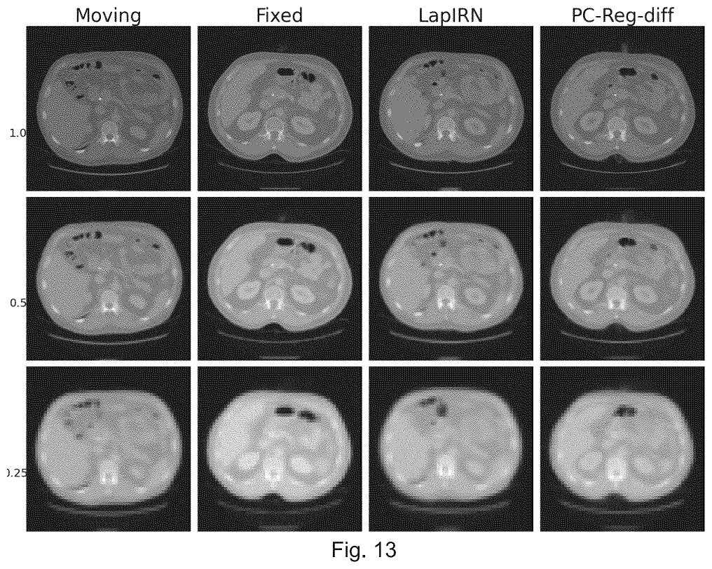

To have a clear comparison between the proposed PC-Reg-diff and the counterpart multi-scale method, LapIRN, we visualize the deformation results at each scale in FIG. 13. FIG. 13 shows qualitative comparison between PC-Reg-diff and LapIRN in each scale on the ABD50 dataset. Full resolution, ½, and ¼ results are shown from top to bottom. PC-Reg achieves better performance from the lowest scale and has a better final prediction. PC-Reg-diff consistently has a better performance in each scale. This implies that PC-Reg-diff is able to reduce the accumulated integration error and achieves the best performance.

### Inference Time

In Table 5, we report the average inference time of each method on ABD50. We perform inference on one test pair from ABD50 for 10 times and calculate the average inference time in seconds. Learning-based methods have superior inference speed compared to conventional methods. Also, PC-Reg can predict the deformation vector field within one second.

### Additional Information

The following disclosure presents additional background and related work that may be useful for the provision of context to the present disclosure.

### Deformable Image Registration

Supervised Methods Deep learning has boosted the research of medical imaging registration. Early supervised methods ((Miao et al., 2016; Cao et al., 2017; Sokooti et al., 2017; Eppenhof and Pluim, 2018)) have achieved good results by using convolution neural networks. However, supervised methods commonly require laborious manual annotation. Hence, most methods either use the deformation vector field obtained by classical methods on intensity image and segmentation masks (Cao et al., 2017) as ground-truth or generate the training pairs by applying synthetic transformations that can be used as target labels (Sokooti et al., 2017; Eppenhof and Pluim, 2018; Yin et al., 2022). Beekman et al. (2021) proposed a learning-based diffeomorphic registration approach in a supervised manner. These methods are limited by the performance of the chosen classical methods or the quality of synthetic transformations. Therefore, the generated ground truth is an upper bound of the model, which does not fully reflect the inherent geometric deformation in image pairs.

The present disclosure is focused on unsupervised image registration. In addition, experiments are conducted with a Dice loss based on the segmentation masks for semi-supervised training.

Unsupervised Methods In order to learn the deformation directly from the data, unsupervised methods commonly use neural networks to estimate the displacement field and transform the moving image to the fixed image by spatial transformers (Jaderberg et al., 2015). Then, a similarity function is used to evaluate the transformation quality that enables end-to-end training. Often regularization terms are added to the similarity term to obtain plausible results. The most used similarity metrics are mean squared error (MSE) (Beg et al., 2005), sum of squared differences (SSD) (Wolberg and Zokai, 2000), normalized cross-correlation (NCC) (Avants et al., 2008), mutual information (MI) (Viola and Wells III, 1997), and modality independent neighborhood descriptor (MIND) (Heinrich et al., 2012). DIRNet (Vos et al., 2017) proposed a patch-based un-supervised framework where a B-spline transformer is used. De Vos et al. (2019) further extend this work by cascading multiple transformer networks together, which is better on large displacement. These models are based on control points transformation (B-spline).

VoxelMorph (Balakrishnan et al., 2019) proposed to directly predict the dense vector field. The framework can be easily extended to efficient diffeomorphic registration (Dalca et al., 2019) by using the scaling and squaring technique (Arsigny et al., 2006). VoxelMorph uses a U-Net (Ronneberger et al., 2015) architecture for single-scale prediction. Trans-Morph (Chen et al., 2022) improves VoxelMorph by using Swin Transformer (Liu et al., 2021) as the basic block of U-Net and achieves state-of-the-art performance. Xmorph (Shi et al., 2022) also uses Transformer blocks and adopts cross-attention to fuse the feature of moving and fixed images. To better model the large deformation, (Mok and Chung, 2020) learns the registration networks in different scales and cascades them together. Dual-PRNet (Hu et al., 2019) also adopts the multi-scale de-sign. Different from LapIRN, it predicts the displacement vector field based on the features of moving and fixed images in each scale. In addition, cycle-consistency constraint (Kim et al., 2021) can be added to improve the training of registration networks. The cycle consistency enhances image registration performance by providing an implicit regularization to preserve topology during the deformation.

Examples of the present disclosure employ multi-scale prediction to address the large deformation problem while maintaining good performance on small deformation. The deformation vector field prediction per scale of the present disclosure is based on the features extracted by a neural network rather than pyramidal images. Different from LapIRN and Dual-PRNet, methods of the present disclosure use a correction network to alleviate the accumulated error in each scale, which reduces integration error in diffeomorphic registration. In addition, unlike Dual-PRNet which only uses displacement vector field parameterization, method according to the present disclosure can be implemented for diffeomorphic registration with a good performance.

Optical Flow Learning-based optical flow methods can also be used to solve the medical image registration task. Optical flow seeks the dense correspondence between two images. FlowNet (Dosovitskiy et al., 2015) is the first work that introduces neural networks into optical flow estimation. Also, it introduced a cross-correlation module which became the fundamental component in the following works. PWC-Net (Sun et al., 2018) improved FlowNet by pyramidal processing, where warping and cost volume are computed in each feature pyramid level. It iteratively predicts and refines the flow field from coarse to fine. The number of PWC-Net refinement iterations is limited by the number of scales. Thus, RAFT (Teed and Deng, 2020) proposed to use a recurrent network to update the flow field in many iterations. RAFT uses a recurrent scheme to iteratively refine the flow field in ⅛ scale. The pyramidal models have the problem of accumulated error due to the multiple bilinear upsampling. To this end, UPFlow (Luo et al., 2021) proposed a self-guided upsample module and a pyramid distillation loss to address the interpolation blur problem caused by bilinear upsampling between pyramid levels.

In medical image registration, the ⅛ scale prediction will lose detailed complex anatomical structure information. Also, the recurrence at a fine-scale level will bring high computation costs in 3D images. Hence, methods according to the present disclosure estimate the deformation vector field in a pyramidal manner which is similar to PWC-Net (Sun et al., 2018). Different from PWC-Net (Sun et al., 2018), the methods of the present disclosure are for more challenging 3D medical images with complex anatomical deformation. Also, PWC-Net does not integrate the intermediate outputs into the final prediction, methods of the present disclosure accumulate the predicted vector field from each scale for modeling the large deformation, and additionally use a correction module to alleviate the accumulated upsampling error. Finally, methods according to the present disclosure can be implemented for pyramidal diffeomorphic registration, which is not considered in PWC-Net.

### Vision Transformer

Transformer (Vaswani et al., 2017) was developed to model sequential data in the natural language processing (NLP) area. The basic building block in a transformer is the self-attention module, which is capable of modelling the long-range relation in sequential data. Vision Transformer (ViT) (Dosovitskiy et al., 2020) extends this concept into the vision community. ViT splits the whole image into a sequence of patches and employs self-attention to replace the convolution. Based on this, Swin Transformer (Liu et al., 2021) proposed a window-based self-attention module and shift window mechanism to produce the feature maps hierarchically. Recently, Transformer has been used in various medical imaging tasks, including segmentation (Chen et al., 2021; Xie et al., 2021), and registration (Liu et al., 2021; Shi et al., 2022). Methods according to the present disclosure may use Swin Transformer (Cao et al., 2021; Chen et al., 2022) blocks in 3D as a feature extractor.

### REFERENCES

Arsigny, V., Commowick, O., Pennec, X., Ayache, N., 2006. A log-euclidean framework for statistics on diffeomorphisms, in: International Conference on Medical Image Computing and Computer-Assisted Intervention, Springer. pp. 924-931.
Ashburner, J., 2007. A fast diffeomorphic image registration algorithm. Neuroimage 38, 95-113.
Avants, B. B., Epstein, C. L., Grossman, M., Gee, J. C., 2008. Symmetric diffeomorphic image registration with cross-correlation: evaluating automated labeling of elderly and neurodegenerative brain. Medical image analysis 12, 26-41.
Avants, B. B., Tustison, N. J., Song, G., Cook, P. A., Klein, A., Gee, J. C., 2011. A reproducible evaluation of ants similarity metric performance in brain image registration. Neuroimage 54, 2033-2044.
Balakrishnan, G., Zhao, A., Sabuncu, M. R., Guttag, J., Dalca, A. V., 2019. Vox-elmorph: a learning framework for deformable medical image registration. IEEE transactions on medical imaging 38, 1788-1800.
Beekman, C., Schaake, E., Sonke, J. J., Remeijer, P., 2021. Deformation trajectory prediction using a neural network trained on finite element data-application to library of ctvs creation for cervical cancer. Physics in Medicine & Biology 66, 215004.
Beg, M. F., Miller, M. I., Trouvé, A., Younes, L., 2005. Computing large de-formation metric mappings via geodesic flows of diffeomorphisms. International journal of computer vision 61, 139-157.
Butcher, J. C., 2016. Numerical methods for ordinary differential equations. John Wiley & Sons.
Cao, H., Wang, Y., Chen, J., Jiang, D., Zhang, X., Tian, Q., Wang, M., 2021. Swin-unet: Unet-like pure transformer for medical image segmentation. arXiv preprint arXiv:2105.05537.
Cao, X., Yang, J., Zhang, J., Nie, D., Kim, M., Wang, Q., Shen, D., 2017. Deformable image registration based on similarity-steered cnn regression, in: International Conference on Medical Image Computing and Computer-Assisted Intervention, Springer. pp. 300-308.
Cao, Y., Miller, M. I., Winslow, R. L., Younes, L., 2005. Large deformation diffeomorphic metric mapping of vector fields. IEEE transactions on medical imaging 24, 1216-1230.
Chen, J., Frey, E. C., He, Y., Segars, W. P., Li, Y., Du, Y., 2022. Transmorph: Transformer for unsupervised medical image registration. Medical Image Analysis 82, 102615.
Chen, J., Lu, Y., Yu, Q., Luo, X., Adeli, E., Wang, Y., Lu, L., Yuille, A. L., Zhou, Y., 2021. Transunet: Transformers make strong encoders for medical image segmentation. arXiv preprint arXiv:2102.04306.
Dalca, A. V., Balakrishnan, G., Guttag, J., Sabuncu, M. R., 2019. Unsupervised learning of probabilistic diffeomorphic registration for images and surfaces. Medical image analysis 57, 226-236.
De Vos, B. D., Berendsen, F. F., Viergever, M. A., Sokooti, H., Staring, M., Is\check{ } gum, I., 2019. A deep learning framework for unsupervised affine and deformable image registration. Medical image analysis 52, 128-143.
Dice, L. R., 1945. Measures of the amount of ecologic association between species. Ecology 26, 297-302.
Dosovitskiy, A., Beyer, L., Kolesnikov, A., Weissenborn, D., Zhai, X., Un-terthiner, T., Dehghani, M., Minderer, M., Heigold, G., Gelly, S., Uszkoreit, J., Houlsby, N., 2020. An image is worth 16×16 words: Transformers for image recognition at scale. arXiv preprint arXiv:2010.11929.
Dosovitskiy, A., Fischer, P., IIg, E., Hausser, P., Hazirbas, C., Golkov, V., Van Der Smagt, P., Cremers, D., Brox, T., 2015. Flownet: Learning optical flow with convolutional networks, in: Proceedings of the IEEE international conference on computer vision, pp. 2758-2766.
Eppenhof, K. A., Pluim, J. P., 2018. Pulmonary ct registration through supervised learning with convolutional neural networks. IEEE transactions on medical imaging 38, 1097-1105.
Fischl, B., 2012. Freesurfer. Neuroimage 62, 774-781.
Heinrich, M. P., Hansen, L., 2020. Highly accurate and memory efficient unsupervised learning-based discrete ct registration using 2.5 d displacement search, in: International Conference on Medical Image Computing and Computer-Assisted Intervention, Springer. pp. 190-200.
Heinrich, M. P., Jenkinson, M., Bhushan, M., Matin, T., Gleeson, F. V., Brady, M., Schnabel, J. A., 2012. Mind: Modality independent neighbourhood descriptor for multi-modal deformable registration. Medical image analysis 16, 1423-1435.
Hering, A., Hansen, L., Mok, T. C. W., Chung, A. C. S., Siebert, H., Ha\ddot{ } ger, S., Lange, A., Kuckertz, S., Heldmann, S., Shao, W., Vesal, S., Rusu, M., Sonn, G., Estienne, T., Vakalopoulou, M., Han, L., Huang, Y., Yap, P. T., Brudfors, M., Balbastre, Y., Joutard, S., Modat, M., Lifshitz, G., Raviv, D., Lv, J., Li, Q., Jaouen, V., Visvikis, D., Fourcade, C., Rubeaux, M., Pan, W., Xu, Z., Jian, B., De Benetti, F., Wodzinski, M., Gunnarsson, N., Sjo\ddot{ } Iund, J., Grzech, D., Qiu, H., Li, Z., Thorley, A., Duan, J., Grofßbro\ddot{ } hmer, C., Hoopes, A., Reinertsen, I., Xiao, Y., Landman, B., Huo, Y., Murphy, K., Lessmann, N., Van Ginneken, B., Dalca, A. V., Heinrich, M. P., 2022. Learn2reg: comprehensive multi-task medical image registration challenge, dataset and evaluation in the era of deep learning. IEEE Transactions on Medical Imaging.
Hu, X., Kang, M., Huang, W., Scott, M. R., Wiest, R., Reyes, M., 2019. Dual-stream pyramid registration network, in: International Conference on Medical Image Computing and Computer-Assisted Intervention, Springer. pp. 382-390.
Huang, G., Liu, Z., Van Der Maaten, L., Weinberger, K. Q., 2017. Densely connected convolutional networks, in: Proceedings of the IEEE conference on computer vision and pattern recognition, pp. 4700-4708.
Jaderberg, M., Simonyan, K., Zisserman, A., Kavukcuoglu, K., 2015. Spatial transformer networks. Advances in neural information processing systems 28.
Kim, B., Kim, D. H., Park, S. H., Kim, J., Lee, J. G., Ye, J. C., 2021. Cyclemorph: cycle consistent unsupervised deformable image registration. Medical Im age Analysis 71, 102036.
Kingma, D. P., Ba, J., 2014. Adam: A method for stochastic optimization. arXiv preprint arXiv:1412.6980.
Klein, S., Staring, M., Murphy, K., Viergever, M. A., Pluim, J. P., 2009. Elastix: a toolbox for intensity-based medical image registration. IEEE transactions on medical imaging 29, 196-205.
Liu, Z., Lin, Y., Cao, Y., Hu, H., Wei, Y., Zhang, Z., Lin, S., Guo, B., 2021. Swin transformer: Hierarchical vision transformer using shifted windows, in: Proceedings of the IEEE/CVF International Conference on Computer Vision, pp. 10012-10022.
Lu, Y., Valmadre, J., Wang, H., Kannala, J., Harandi, M., Torr, P., 2020. Devon: Deformable volume network for learning optical flow, in: Proceedings of the IEEE/CVF Winter Conference on Applications of Computer Vision, pp. 2705-2713.
Luo, K., Wang, C., Liu, S., Fan, H., Wang, J., Sun, J., 2021. Upflow: Up sampling pyramid for unsupervised optical flow learning, in: Proceedings of the IEEE/CVF Conference on Computer Vision and Pattern Recognition, pp. 1045-1054.
Ma, J., Zhang, Y., Gu, S., Zhu, C., Ge, C., Zhang, Y., An, X., Wang, C., Wang, Q., Liu, X., Cao, S., Zhang, Q., Liu, S., Wang, Y., Li, Y., He, J., Yang, X., 2021. Abdomenct-1 k: Is abdominal organ segmentation a solved problem? IEEE Transactions on Pattern Analysis and Machine Intelligence doi:10.1109/TPAMI.2021.3100536.
Marcus, D. S., Wang, T. H., Parker, J., Csernansky, J. G., Morris, J. C., Buckner, R. L., 2007. Open access series of imaging studies (oasis): cross-sectional mri data in young, middle aged, nondemented, and demented older adults. Journal of cognitive neuroscience 19, 1498-1507.
Miao, S., Wang, Z. J., Liao, R., 2016. A cnn regression approach for real-time 2d/3d registration. IEEE transactions on medical imaging 35, 1352-1363.
Modat, M., Ridgway, G. R., Taylor, Z. A., Lehmann, M., Barnes, J., Hawkes, D. J., Fox, N. C., Ourselin, S., 2010. Fast free-form deformation using graphics processing units. Computer methods and programs in biomedicine 98, 278-284.
Mok, T. C., Chung, A., 2020. Large deformation diffeomorphic image registration with Iaplacian pyramid networks, in: International Conference on Medical Image Computing and Computer-Assisted Intervention, Springer. pp. 211-221.
Pegios, P., Czolbe, S., 2022. Can transformers capture long-range displacements better than cnns?, in: Medical Imaging with Deep Learning.
Ronneberger, O., Fischer, P., Brox, T., 2015. U-net: Convolutional networks for biomedical image segmentation, in: International Conference on Medical image computing and computer-assisted intervention, Springer. pp. 234-241.
Ru\ddot{ } haak, J., Polzin, T., Heldmann, S., Simpson, I. J., Handels, H., Modersitzki, J., Heinrich, M. P., 2017. Estimation of large motion in lung ct by integrating regularized keypoint correspondences into dense deformable registration. IEEE transactions on medical imaging 36, 1746-1757.
Shi, J., He, Y., Kong, Y., Coatrieux, J. L., Shu, H., Yang, G., Li, S., 2022. Xmorpher: Full transformer for deformable medical image registration via cross attention. arXiv preprint arXiv:2206.07349.
Sokooti, H., Vos, B. d., Berendsen, F., Lelieveldt, B. P., Is\check{ } gum, I., Staring, M., 2017. Nonrigid image registration using multi-scale 3d convolutional neural networks, in: International conference on medical image computing and computer-assisted intervention, Springer. pp. 232-239.
Sun, D., Yang, X., Liu, M. Y., Kautz, J., 2018. Pwc-net: Cnns for optical flow using pyramid, warping, and cost volume, in: Proceedings of the IEEE conference on computer vision and pattern recognition, pp. 8934-8943.
Teed, Z., Deng, J., 2020. Raft: Recurrent all-pairs field transforms for optical flow, in: European conference on computer vision, Springer. pp. 402-419.
Vaswani, A., Shazeer, N., Parmar, N., Uszkoreit, J., Jones, L., Gomez, A. N., Kaiser, L., Polosukhin, I., 2017. Attention is all you need. Advances in neural information processing systems 30.
Viola, P., Wells Ill, W. M., 1997. Alignment by maximization of mutual information. International journal of computer vision 24, 137-154.
Vos, B. D. d., Berendsen, F. F., Viergever, M. A., Staring, M., Is\check{ } gum, I., 2017. End-to-end unsupervised deformable image registration with a convolutional neural network, in: Deep learning in medical image analysis and multimodal learning for clinical decision support. Springer, pp. 204-212.
Wolberg, G., Zokai, S., 2000. Robust image registration using log-polar trans form, in: Proceedings 2000 International Conference on Image Processing (Cat. No. 00CH37101), IEEE. pp. 493-496.
Xie, Y., Zhang, J., Shen, C., Xia, Y., 2021. Cotr: Efficiently bridging cnn and transformer for 3d medical image segmentation, in: International conference on medical image computing and computer-assisted intervention, Springer. pp. 171-180.
Yin, W., Chagin, V. O., Cardoso, M. C., Rohr, K., 2022. Non-rigid registration of temporal live cell microscopy image sequences using deep learning, in: Medical Imaging 2022: Image Processing, SPIE. pp. 353-358.

What is claimed is:

1. A computer implemented method for performing deformable image registration on first volumetric medical image and a second volumetric medical image, the method comprising:
(i) for each of the first volumetric medical image and the second volumetric medical image, extracting features from the first volumetric medical image and the second volumetric medical image at each of a plurality of different scales;
(ii) initiating a mapping between the first volumetric medical image and the second volumetric medical image at a lowest of the plurality of different scales;
(iii) sequentially, for each scale of the plurality of different scales that is above the lowest scale:
predicting a mapping between the first volumetric medical image and the second volumetric medical image at a given scale using a deformation field and features extracted from the first volumetric medical image and the second volumetric medical image at a scale immediately below the given scale; and
correcting the predicted mapping between the first volumetric medical image and the second volumetric medical image at the given scale using features extracted from the first volumetric medical image and the second volumetric medical image at the given scale;
the method further comprising:
(iv) predicting the deformation field between the first volumetric medical image and the second volumetric medical image at full resolution using the corrected prediction of the mapping at a highest of the plurality of different scales.

2. The method as claimed in claim 1, wherein the mapping between the first volumetric medical image and the second volumetric medical image comprises at least one of:
a deformation field between the first volumetric medical image and the second volumetric medical image; or
a velocity field between the first volumetric medical image and the second volumetric medical image.

3. The method as claimed in claim 1, wherein predicting the mapping between the first volumetric medical image and the second volumetric medical image at a given scale using the deformation field and features extracted from the first volumetric medical image and the second volumetric medical image at the scale immediately below the given scale comprises:
performing trilinear upsampling of the mapping at the scale immediately below the given scale;
generating a predicted residual vector field for the given scale using a prediction machine learning (ML) model; and
generating a predicted mapping for the given scale by combining a result of the trilinear upsampling with the generated predicted residual vector field.

4. The method as claimed in claim 3, wherein the prediction ML model implements a function of the mapping and of features extracted from the first volumetric medical image and the second volumetric medical image at the scale immediately below the given scale.

5. The method as claimed in claim 3, wherein generating a residual vector field using the prediction machine learning, ML, model comprises:
generating an input to the prediction ML model by using the deformation field at the scale immediately below the given scale to warp the features extracted from the second volumetric medical image at the scale immediately below the given scale, and concatenating the warped features with the features extracted from the first volumetric medical image at the scale immediately below the given scale; and
causing the prediction ML model to process the generated input and to generate an output comprising the predicted residual vector field.

6. The method as claimed in claim 3, wherein the prediction ML model comprises a convolutional neural network having a transposed convolution layer.

7. The method as claimed in claim 3, wherein correcting the predicted mapping between the first volumetric medical image and the second volumetric medical image at the given scale using features extracted from the first volumetric medical image and the second volumetric medical image at the given scale comprises:
generating a corrected residual vector field using a correction ML model; and
generating a corrected mapping for the given scale by combining the result of the trilinear upsampling with a function of the predicted residual vector field and the corrected residual vector field.

8. The method as claimed in claim 7, wherein the function of the predicted residual vector field and the corrected residual vector field comprises an average of the predicted residual vector field and the corrected residual vector field.

9. The method as claimed in claim 7, wherein the correction ML model implements a function of the predicted mapping for the given scale and features extracted from the first volumetric medical image and the second volumetric medical image at the given scale.

10. The method as claimed in claim 7, wherein generating the corrected residual vector field using the correction ML model comprises:
generating an input to the correction ML model by using the predicted mapping at the given scale to warp the features extracted from the second volumetric medical image at the given scale, and concatenating the warped features with the features extracted from the first volumetric medical image at the given scale; and
causing the correction ML model to process the generated input and to generate an output comprising the corrected residual vector field.

11. The method as claimed in claim 10, wherein predicting the mapping between the first volumetric medical image and the second volumetric medical image at a given scale using the deformation field and features extracted from the first volumetric medical image and the second volumetric medical image at the scale immediately below the given scale comprises:
performing trilinear upsampling of the mapping at the scale immediately below the given scale;
generating a predicted residual vector field for the given scale using a prediction machine learning, ML, model; and
generating a predicted mapping for the given scale by combining the result of the trilinear upsampling with the generated predicted residual vector field, wherein the predicted mapping between the first volumetric medical image and the second volumetric medical image at a given scale comprises the prediction following trilinear upsampling of the mapping at the scale immediately below the given scale.

12. The method as claimed in claim 7, wherein the correction ML model comprises a convolutional neural network without a transposed convolution layer.

13. The method as claimed in claim 1, wherein the mapping between the first volumetric medical image and the second volumetric medical image comprises a velocity field between the first volumetric medical image and the second volumetric medical image, and wherein the method further comprises, for each scale of the plurality of different scales:
integrating the corrected prediction of the velocity field between the first volumetric medical image and the second volumetric medical image to generate a corrected prediction of a deformation field at the given scale.

14. The method as claimed in claim 13, wherein predicting the mapping between the first volumetric medical image and the second volumetric medical image at a given scale using the deformation field and features extracted from the first and second volumetric medical images at the scale immediately below the given scale comprises:
performing trilinear upsampling of the mapping at the scale immediately below the given scale;
generating a predicted residual vector field for the given scale using a prediction machine learning, ML, model; and
generating a predicted mapping for the given scale by combining a result of the trilinear upsampling with the generated predicted residual vector field;
wherein correcting the predicted mapping between the first volumetric medical image and the second volumetric medical image at the given scale using features extracted from the first volumetric medical image and the second volumetric medical image at the given scale comprises:
generating a corrected residual vector field using a correction ML model; and
generating a corrected mapping for the given scale by combining the result of the trilinear upsampling with a function of the predicted residual vector field and the corrected residual vector field;
wherein generating the corrected residual vector field using the correction ML model comprises:
generating an input to the correction ML model by using the predicted mapping at the given scale to warp the features extracted from the second volumetric medical image at the given scale, and concatenating the warped features with the features extracted from the first volumetric medical image at the given scale; and
causing the correction ML model to process the generated input and to generate an output comprising the corrected residual vector field;
wherein using the predicted mapping at the given scale to warp the features extracted from the second volumetric medical image at the given scale comprises:
integrating the predicted velocity field at the given scale to obtain a predicted deformation field at the given scale, and
using the predicted deformation field at the given scale to warp the features extracted from the second image at the given scale.

15. The method as claimed in claim 1, further comprising:
during a training period:
repeating steps (i)-(iv) for a plurality of pairs of first and second volumetric medical images; and
updating parameters of the prediction and correction steps to minimize a loss function, wherein the loss function comprises a distillation loss component, and wherein the distillation loss component comprises a loss between the predicted deformation field between the first and second volumetric medical images of a pair at full resolution, and the predicted deformation field between the first and second volumetric medical images of the pair at a given scale.

16. The method as claimed in claim 15, further comprising:
updating parameters of an ML model for extracting features from the first volumetric medical image and the second volumetric medical image at each of the plurality of different scales.

17. The method as claimed in claim 15, wherein the loss function further comprises a similarity loss component and a regularization loss component.

18. The method as claimed in claim 17, wherein the similarity loss component comprises an aggregation of similarity loss at each of the plurality of different scales.

19. The method as claimed in claim 1, wherein, for each of the first volumetric medical image and the second volumetric medical image, extracting features from the first volumetric medical image and the second volumetric medical image at each of a plurality of different scales comprises, for an image:
partitioning the image into a plurality of volumetric patches; and
generating a hierarchical feature map using a self-attention based ML model.

20. The method as claimed in claim 1, further comprising:
using a transformer ML model to extract features from the first volumetric medical image and the second volumetric medical image at each of a plurality of different scales.

21. A computer implemented method for adaptation of a reference Radiotherapy (RT) treatment plan, wherein the reference RT treatment plan is associated with a first volumetric medical image of a patient, the method comprising:
acquiring a second volumetric medial image of a patient;
performing deformable image registration of the first volumetric medical image and the second volumetric medical image; and
using a predicted deformation field between the first volumetric medical image and the second volumetric medical images at full resolution to adapt the reference treatment plan.

22. A computer program product comprising a non-transitory computer readable medium, the non-transitory computer readable medium having computer readable code embodied therein, the computer readable code being configured such that, when performed by a computer or a processor, the computer or the processor is caused to:
(i) for each of a first volumetric medical image and a second volumetric medical image, extract features from the images at each of a plurality of different scales;
(ii) initiate a mapping between the first volumetric medical image and the second volumetric medical image at a lowest of the plurality of different scales;
(iii) sequentially, for each scale of the plurality of different scales that is above the lowest scale:
predict a mapping between the first volumetric medical image and the second volumetric medical image at a given scale using a deformation field and features extracted from the first volumetric medical image and the second volumetric medical image at a scale immediately below the given scale; and
correct the predicted mapping between the first volumetric medical image and the second volumetric medical image at the given scale using one or more features extracted from the first volumetric medical image and second volumetric medical image at the given scale;
(iv) predict the deformation field between the first volumetric medical image and the second volumetric medical image at full resolution using the corrected prediction of the mapping at a highest of the plurality of different scales.

23. A registration node for performing deformable image registration on first volumetric medical image and a second volumetric medical image, the registration node comprising processing circuitry configured to cause the registration node to:
(i) for each of the first volumetric medical image and the second volumetric medical image, extract features from the images at each of a plurality of different scales;
(ii) initiate a mapping between the first volumetric medical image and the second volumetric medical image at a lowest of the plurality of different scales;
(iii) sequentially, for each scale of the plurality of different scales that is above the lowest scale:
predict a mapping between the first volumetric medical image and the second volumetric medical image at a given scale using a deformation field and features extracted from the first volumetric medical image and the second volumetric medical image at the scale immediately below the given scale; and
correct the predicted mapping between the first volumetric medical image and the second volumetric medical image at the given scale using features extracted from the first and second volumetric medical images at the given scale;
wherein the processing circuitry is further configured to cause the registration node to:
(iv) predict the deformation field between the first volumetric medical image and the second volumetric medical image at full resolution using the corrected prediction of the mapping at a highest of the plurality of different scales.

24. The registration node of claim 23, wherein the registration node is included in a radiotherapy treatment apparatus.

25. A planning node for adapting a reference Radiotherapy (RT) treatment plan, wherein the reference RT treatment plan is associated with a first volumetric medical image of a patient, and wherein the planning node comprising processing circuitry configured to cause the planning node to:
acquire a second volumetric medial image of a patient;
perform deformable image registration of the first and second volumetric medical images; and
use a predicted deformation field between the first volumetric medical image and the second volumetric medical image at full resolution to adapt the reference treatment plan.

26. The planning node of claim 25, wherein the planning node is included in a radiotherapy treatment apparatus.
